(19) United States
(12) Patent Application Publication
(10) Pub. No.: US 2026/0356803 A1
(43) Pub. Date: Nov. 12, 2026

(54) Nicotinamide derivatives

(71) Applicant: Board of Regents, The University of Texas System, Austin, TX (US)

(72) Inventors: Jef De Brabander, Dallas, TX (US); Deepak Nijhawan, Dallas, TX (US); Wentian Wang, Dallas, TX (US); Panayotis Christos Theodoropoulos, Dallas, TX (US); Holly Huizi Guo, Dallas, TX (US)

(73) Assignee: Board of Regents, The University of Texas System, Austin, TX (US)

(21) Appl. No.: 19/736,982

(22) Filed: Jul. 10, 2026

Related U.S. Application Data
(63) Continuation of application No. PCT/US25/10002, filed on Jan. 1, 2025.
(60) Provisional application No. 63/619,374, filed on Jan. 10, 2024.

(51) Int. Cl. C07D 213/75; A61K 31/4406; A61K 31/443; A61K 31/4436; A61K 31/4439; A61K 31/444; A61K 31/455; A61K 31/501; A61P 35/00; C07D 213/81; C07D 213/82; C07D 401/12; C07D 405/12; C07D 409/12; C07D 413/12; C07D 417/12; C07D 513/04

(52) U.S. Cl. CPC C07D 213/75; A61K 31/4406; A61K 31/443; A61K 31/4436; A61K 31/4439; A61K 31/444; A61K 31/455; A61K 31/501; A61P 35/00; C07D 213/81; C07D 213/82; C07D 401/12; C07D 405/12; C07D 409/12; C07D 413/12; C07D 417/12; C07D 513/04

(57) ABSTRACT

Compounds and pharmaceutical compositions comprising nicotinamide derivatives are used to treat neuronal cancers.

### CROSS-REFERENCES TO RELATED APPLICATIONS

This application is a continuation of PCT/US25/10002, filed Jan. 1, 2025, which claims priority to U.S. Provisional Application No. 63/619,374, filed Jan. 10, 2024, the disclosure of which is hereby incorporated by reference in its entirety for all purposes.

### INTRODUCTION

The prognosis for patients with peripheral and central nervous system cancers, such as malignant peripheral nerve sheath tumors (MPNST) and glioblastoma (GBM) remains dismal and new treatments are needed. MPNST is more likely to affect younger patients, with median age of diagnosis at 42 years old and median overall survival of 8 years.¹ On the other hand, GBM tends to affect older patients and has worse prognosis with a median overall survival after standard of care treatment of 14.6 months.^{2,3}

Compound 21, an N-pyridinylthiophene carboxamide, was identified as a potential pre-clinical lead for the treatment of GBM and MPNST.⁴ Identified as a hit in a cell-based screen of 200,000 synthetic compounds for anti-proliferative activity in primary mouse GBM cells, compound 21 was subsequently shown to be selectively toxic to a variety of mouse and human MPNST cell lines and nontoxic to Schwann cells and mouse embryonic fibroblasts. Further, compound 21 was bioavailable when dosed orally and inhibited the growth of human MPNST cells subcutaneously xenografted into mice, without obvious toxic effects. However, the compound's mechanism of action remains unknown. We reasoned that elucidating this might identify a new therapeutic strategy for these difficult-to-treat cancers.

### SUMMARY OF THE INVENTION

The invention provides nicotinamide derivatives for use in neuronal cancers. In aspects and embodiments the invention provides:
1. A compound of formula I:

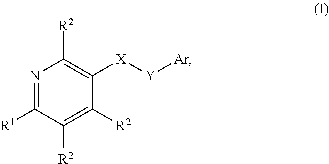

wherein:
X—Y is C(O)NH or NHC(O);
R1 is selected from H, D, F, Cl, Br, CN;
each R2 is independently selected from H or D;
Ar is selected from Ph, fused phenyl, heteroaryl, fused heteroaryl including but not limited to thiophenes, oxazoles, isoxazoles, thiazoles, isothiazoles, furans, pyrroles, pyrazoles, imidazoles, triazoles, tetrazoles, pyridines, pyrimidines, pyrazines, pyridazines, naphtyls, quinolines, isoquinolines, quinazolines, quinoxalines, cinnolines, phtalazines, naphthyridines, pyridopyrazines, pyrazolopyridines, pyridopyrimidines, pyridopyridazines, benzoxazoles, tetrahydrobenzoxazoles, benzoisoxazoles, tetrahydrobenzoisoxazoles, benzothiazoles, tetrahydrobenzothiazoles, benzoisothiazoles, tetrahydrobezoisothiazoles, indoles, tetrahydroindoles, indazoles, benzofurans, tetrahydrofurans, benzothiophenes, tetrahydrobenzothiphenes, imidazopyridines, benzodioxoles, benzoxazolones, benzoisoxazolones, benzothiazolones, benzoisothiazolones, 1,3-dihydro-2H-benzimidazoles, benzo[d]imidazol-2-ones, benzodioxolones, dioxolopyridines, oxazolopyridines, oxazolopyrimidines, oxazolopyrazines, oxazolopyridazines, isoxazolopyridines, isoxazolopyrimidines, isoxazolopyrazines, isoxazolopyridazines, thiazolopyridines, thiazolopyrimidines, thiazolopyrazines, thiazolopyridazines, imidazopyridines, thienopyridines, thienopyridazines, thienopyrimidines, thienopyrazines, furopyridines, furopyrazines, furopyrimidines, furopyridazines, pyrrolopyridines, pyrrolopyrimidines, pyrrolopirazines, pyrrolopyridazines, pyrazolopyridines, pyrazolopyrimidines, pyrazolopyrazines, pyrazolopyridazines, dioxolopyridazines, dioxolopyrimidines, dioxolopyrazines, coumarins, isocoumarins and all possible isomers thereof, each optionally substituted at any position with: D, F, Cl, Br, CN, OH, aryl, heteroaryl, R3, OR3, C(O)R3, CO2R3;
R3 is selected from C1-C3alkyl, cPr, cBu, oxetanyl, C1-C3alkynyl, C1-C3alkenyl, each optionally substituted with a substituent selected from D, F, CN, OH, OR4; and
R4 is selected from Me, CD3, CF3, CHF2, CH2F, cPr, oxetanyl;
wherein the compound and composition claims herein exclude compound 21

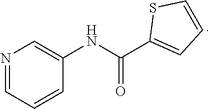 [SMILES: O=C(Nc1cccnc1)c1cccs1]

2. A compound of claim 1, wherein:
Ar is selected from Ph, thiophene, furan, pyrrole and all possible isomers thereof, each optionally substituted at any position with a substituent selected from D, F, Cl, CN, OMe, OH, OCD3, OCF3, OCHF2, OCH2F, CH2OH, CH2OMe, CH2OCD3, CH2OCF3, CH2OCHF2, CH2OCH2F.
3. A compound of claim 1, wherein:
Ar is thiophene.
4. A compound of claim 1, wherein:
R1 is H or D; and
Ar is selected from Ph, thiophene, furan, pyrrole and all possible isomers thereof, each optionally substituted at any position with a substituent selected from D, F, Cl, CN, OMe, OH, OCD3, OCF3, OCHF2, OCH2F, CH2OH, CH2OMe, CH2OCD3, CH2OCF3, CH2OCHF2, CH2OCH2F.
5. A compound of claim 1, wherein:
R1 is H or D; and
Ar is thiophene.
6. A compound of claim 1, wherein:
X—Y is C(O)NH;
R1 is H or D; and
Ar is selected from Ph, thiophene, furan, pyrrole and all possible isomers thereof, each optionally substituted at any position with a substituent selected from D, F, Cl, CN, OMe, OH, OCD3, OCF3, OCHF2, OCH2F, CH2OH, CH2OMe, CH2OCD3, CH2OCF3, CH2OCHF2, CH2OCH2F.
7. A compound of claim 1, wherein:
X—Y is C(O)NH;
R1 is H or D; and
Ar is thiophene.
8. A compound of claim 1, wherein:
X—Y is C(O)NH;
R1 is H or D; and
Ar is thiophen-2-yl.
9. A compound of claim 1, selected from Table 1.
10. A compound of claim 1 selected from:

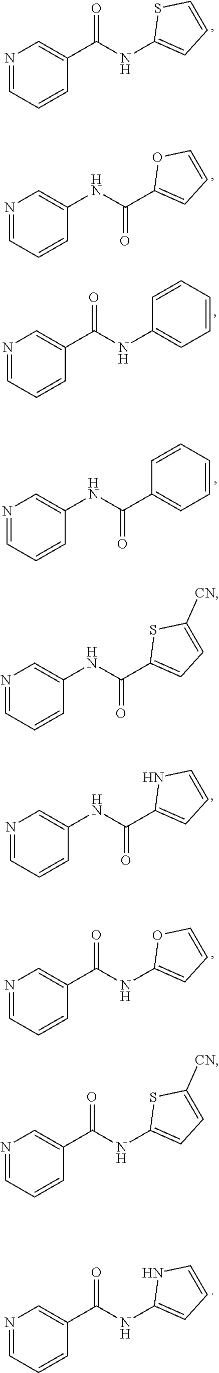

11. A compound of claim 1 of formula:

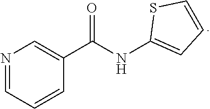 [SMILES: O=C(Nc1cccs1)c1cccnc1]

12. A pharmaceutical composition comprising a compound of claim 1, 2, 3, 4, 5, 6, 7, 8, 9, 10, or 11, or a pharmaceutically acceptable salt, a hydrate or a stereoisomer thereof, and a pharmaceutically acceptable carrier or excipient, in a pharmaceutically acceptable unit dosage.
13. A method to treat or inhibit cancer, particularly a neuronal cancer, including peripheral and central nervous system cancers, such as glioblastoma and malignant peripheral nerve sheath tumors (MPNST), comprising administering to a person in need thereof a compound of claim 1, 2, 3, 4, 5, 6, 7, 8, 9, 10 or 11.
14. A method of claim 13, further comprising the antecedent step of detecting or diagnosing a disease or condition indicating the need thereof.
15. A method of claim 13, further comprising the antecedent step of detecting or diagnosing a disease or condition indicating the need thereof, and the subsequent step of detecting a resultant improvement or delay of progression of the disease or condition.

The invention encompasses all combinations of the particular embodiments recited herein, as if each combination had been laboriously recited.

### DESCRIPTION OF PARTICULAR EMBODIMENTS OF THE INVENTION

Unless contraindicated or noted otherwise, in these descriptions and throughout this specification, the terms “a” and “an” mean one or more, the term “or” means and/or. It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein, including citations therein, are hereby incorporated by reference in their entirety for all purposes.

The term “alkyl” refers to a hydrocarbon group selected from linear and branched saturated hydrocarbon groups of 1-18, or 1-12, or 1-6 carbon atoms. Examples of the alkyl group include methyl, ethyl,1-propyl or n-propyl (“n-Pr”), 2-propyl or isopropyl (“i-Pr”), 1-butyl or n-butyl (“n-Bu”), 2-methyl-1-propyl or isobutyl (“i-Bu”), 1-methylpropyl or s-butyl (“s-Bu”), and 1,1-dimethylethyl or t-butyl (“t-Bu”). Other examples of the alkyl group include 1-pentyl, 2-pentyl, 3-pentyl, 2-methyl-2-butyl, 3-methyl-2-butyl, 3-methyl-1-butyl, 2-methyl-1-butyl, 1-hexyl, 2-hexyl, 3-hexyl, 2-methyl-2-pentyl, 3-methyl-2-pentyl, 4-methyl-2-pentyl, 3-methyl-3-pentyl, 2-methyl-3-pentyl, 2,3-dimethyl-2-butyl and 3,3-dimethyl-2-butyl groups.

Lower alkyl means 1-8, preferably 1-6, more preferably 1-4 carbon atoms; lower alkenyl or alkynyl means 2-8, 2-6 or 2-4 carbon atoms.

The term “alkenyl” refers to a hydrocarbon group selected from linear and branched hydrocarbon groups comprising at least one C═C double bond and of 2-18, or 2-12, or 2-6 carbon atoms. Examples of the alkenyl group may be selected from ethenyl or vinyl, prop-1-enyl, prop-2-enyl, 2-methylprop-1-enyl, but-1-enyl, but-2-enyl, but-3-enyl, buta-1,3-dienyl, 2-methylbuta-1,3-diene, hex-1-enyl, hex-2-enyl, hex-3-enyl, hex-4-enyl, and hexa-1,3-dienyl groups.

The term “alkynyl” refers to a hydrocarbon group selected from linear and branched hydrocarbon group, comprising at least one C≡C triple bond and of 2-18, or 2-12, or 2-6 carbon atoms. Examples of the alkynyl group include ethynyl, 1-propynyl, 2-propynyl (propargyl), 1-butynyl, 2-butynyl, and 3-butynyl groups.

The term “cycloalkyl” refers to a hydrocarbon group selected from saturated and partially unsaturated cyclic hydrocarbon groups, comprising monocyclic and polycyclic (e.g., bicyclic and tricyclic) groups. For example, the cycloalkyl group may be of 3-12, or 3-8, or 3-6 carbon atoms. Even further for example, the cycloalkyl group may be a monocyclic group of 3-12, or 3-8, or 3-6 carbon atoms. Examples of the monocyclic cycloalkyl group include cyclopropyl, cyclobutyl, cyclopentyl, 1-cyclopent-1-enyl, 1-cyclopent-2-enyl, 1-cyclopent-3-enyl, cyclohexyl, 1-cyclohex-1-enyl, 1-cyclohex-2-enyl, 1-cyclohex-3-enyl, cyclohexadienyl, cycloheptyl, cyclooctyl, cyclononyl, cyclodecyl, cycloundecyl, and cyclododecyl groups. Examples of the bicyclic cycloalkyl groups include those having 7-12 ring atoms arranged as a bicycle ring selected from [4,4], [4,5], [5,5], [5,6] and [6,6] ring systems, or as a bridged bicyclic ring selected from bicyclo[2.2.1]heptane, bicyclo[2.2.2]octane, and bicyclo[3.2.2]nonane. The ring may be saturated or have at least one double bond (i.e. partially unsaturated), but is not fully conjugated, and is not aromatic, as aromatic is defined herein.

The term “aryl” herein refers to a group selected from: 5- and 6-membered carbocyclic aromatic rings, for example, phenyl; bicyclic ring systems such as 7-12 membered bicyclic ring systems wherein at least one ring is carbocyclic and aromatic, selected, for example, from naphthalene, indane, and 1,2,3,4-tetrahydroquinoline; and tricyclic ring systems such as 10-15 membered tricyclic ring systems wherein at least one ring is carbocyclic and aromatic, for example, fluorene.

For example, the aryl group is selected from 5- and 6-membered carbocyclic aromatic rings fused to a 5- to 7-membered cycloalkyl or heterocyclic ring optionally comprising at least one heteroatom selected from N, O, and S, provided that the point of attachment is at the carbocyclic aromatic ring when the carbocyclic aromatic ring is fused with a heterocyclic ring, and the point of attachment can be at the carbocyclic aromatic ring or at the cycloalkyl group when the carbocyclic aromatic ring is fused with a cycloalkyl group. Bivalent radicals formed from substituted benzene derivatives and having the free valences at ring atoms are named as substituted phenylene radicals. Bivalent radicals derived from univalent polycyclic hydrocarbon radicals whose names end in “-yl” by removal of one hydrogen atom from the carbon atom with the free valence are named by adding “-idene” to the name of the corresponding univalent radical, e.g., a naphthyl group with two points of attachment is termed naphthylidene. Aryl, however, does not encompass or overlap with heteroaryl, separately defined below. Hence, if one or more carbocyclic aromatic rings are fused with a heterocyclic aromatic ring, the resulting ring system is heteroaryl, not aryl, as defined herein.

The term “halogen” or “halo” refers to F, Cl, Br or I.

The term “heteroalkyl” refers to alkyl comprising at least one heteroatom.

The term “heteroaryl” refers to a group selected from:
5- to 7-membered aromatic, monocyclic rings comprising 1, 2, 3 or 4 heteroatoms selected from N, O, and S, with the remaining ring atoms being carbon;
8- to 12-membered bicyclic rings comprising 1, 2, 3 or 4 heteroatoms, selected from N, O, and S, with the remaining ring atoms being carbon and wherein at least one ring is aromatic and at least one heteroatom is present in the aromatic ring; and
11- to 14-membered tricyclic rings comprising 1, 2, 3 or 4 heteroatoms, selected from N, O, and S, with the remaining ring atoms being carbon and wherein at least one ring is aromatic and at least one heteroatom is present in an aromatic ring.

For example, the heteroaryl group includes a 5- to 7-membered heterocyclic aromatic ring fused to a 5- to 7-membered cycloalkyl ring. For such fused, bicyclic heteroaryl ring systems wherein only one of the rings comprises at least one heteroatom, the point of attachment may be at the heteroaromatic ring or at the cycloalkyl ring.

When the total number of S and O atoms in the heteroaryl group exceeds 1, those heteroatoms are not adjacent to one another. In some embodiments, the total number of S and O atoms in the heteroaryl group is not more than 2. In some embodiments, the total number of S and O atoms in the aromatic heterocycle is not more than 1.

Examples of the heteroaryl group include, but are not limited to, (as numbered from the linkage position assigned priority 1) pyridyl (such as 2-pyridyl, 3-pyridyl, or 4-pyridyl), cinnolinyl, pyrazinyl, 2,4-pyrimidinyl, 3,5-pyrimidinyl, 2,4-imidazolyl, imidazopyridinyl, isoxazolyl, oxazolyl, thiazolyl, isothiazolyl, thiadiazolyl, tetrazolyl, thienyl, triazinyl, benzothienyl, furyl, benzofuryl, benzoimidazolyl, indolyl, isoindolyl, indolinyl, phthalazinyl, pyrazinyl, pyridazinyl, pyrrolyl, triazolyl, quinolinyl, isoquinolinyl, pyrazolyl, pyrrolopyridinyl (such as 1H-pyrrolo[2,3-b]pyridin-5-yl), pyrazolopyridinyl (such as 1H-pyrazolo[3,4-b]pyridin-5-yl), benzoxazolyl (such as benzo[d]oxazol-6-yl), pteridinyl, purinyl, 1-oxa-2,3-diazolyl, 1-oxa-2,4-diazolyl, 1-oxa-2,5-diazolyl, 1-oxa-3,4-diazolyl, 1-thia-2,3-diazolyl, 1-thia-2,4-diazolyl, 1-thia-2,5-diazolyl, 1-thia-3,4-diazolyl, furazanyl, benzofurazanyl, benzothiophenyl, benzothiazolyl, benzoxazolyl, quinazolinyl, quinoxalinyl, naphthyridinyl, furopyridinyl, benzothiazolyl (such as benzo[d]thiazol-6-yl), indazolyl (such as 1H-indazol-5-yl) and 5,6,7,8-tetrahydroisoquinoline.

The term “heterocyclic” or “heterocycle” or “heterocyclyl” refers to a ring selected from 4- to 12-membered monocyclic, bicyclic and tricyclic, saturated and partially unsaturated rings comprising at least one carbon atoms in addition to 1, 2, 3 or 4 heteroatoms, selected from oxygen, sulfur, and nitrogen. “Heterocycle” also refers to a 5- to 7-membered heterocyclic ring comprising at least one heteroatom selected from N, O, and S fused with 5-, 6-, and/or 7-membered cycloalkyl, carbocyclic aromatic or heteroaromatic ring, provided that the point of attachment is at the heterocyclic ring when the heterocyclic ring is fused with a carbocyclic aromatic or a heteroaromatic ring, and that the point of attachment can be at the cycloalkyl or heterocyclic ring when the heterocyclic ring is fused with cycloalkyl.

“Heterocycle” also refers to an aliphatic spirocyclic ring comprising at least one heteroatom selected from N, O, and S, provided that the point of attachment is at the heterocyclic ring. The rings may be saturated or have at least one double bond (i.e. partially unsaturated). The heterocycle may be substituted with oxo. The point of the attachment may be carbon or heteroatom in the heterocyclic ring. A heterocyle is not a heteroaryl as defined herein.

Examples of the heterocycle include, but not limited to, (as numbered from the linkage position assigned priority 1) 1-pyrrolidinyl, 2-pyrrolidinyl, 2,4-imidazolidinyl, 2,3-pyrazolidinyl, 1-piperidinyl, 2-piperidinyl, 3-piperidinyl, 4-piperidinyl, 2,5-piperazinyl, pyranyl, 2-morpholinyl, 3-morpholinyl, oxiranyl, aziridinyl, thiiranyl, azetidinyl, oxetanyl, thietanyl, 1,2-dithietanyl, 1,3-dithietanyl, dihydropyridinyl, tetrahydropyridinyl, thiomorpholinyl, thioxanyl, piperazinyl, homopiperazinyl, homopiperidinyl, azepanyl, oxepanyl, thiepanyl, 1,4-oxathianyl, 1,4-dioxepanyl, 1,4-oxathiepanyl, 1,4-oxaazepanyl, 1,4-dithiepanyl, 1,4-thiazepanyl and 1,4-diazepane 1,4-dithianyl, 1,4-azathianyl, oxazepinyl, diazepinyl, thiazepinyl, dihydrothienyl, dihydropyranyl, dihydrofuranyl, tetrahydrofuranyl, tetrahydrothienyl, tetrahydropyranyl, tetrahydrothiopyranyl, 1-pyrrolinyl, 2-pyrrolinyl, 3-pyrrolinyl, indolinyl, 2H-pyranyl, 4H-pyranyl, 1,4-dioxanyl, 1,3-dioxolanyl, pyrazolinyl, pyrazolidinyl, dithianyl, dithiolanyl, pyrazolidinylimidazolinyl, pyrimidinonyl, 1,1-dioxo-thiomorpholinyl, 3-azabicyco[3.1.0]hexanyl, 3-azabicyclo[4.1.0]heptanyl and azabicyclo[2.2.2]hexanyl. Substituted heterocycle also includes ring systems substituted with one or more oxo moieties, such as piperidinyl N-oxide, morpholinyl-N-oxide, 1-oxo-1-thiomorpholinyl and 1, 1-dioxo-1-thiomorpholinyl.

Substituents are selected from: halogen, —R′, —OR′, ═O, ═NR′, ═N—OR′, —NR′R″, —SR′, —SiR′R″R′″, —OC(O)R′, —C(O)R′, —CO₂R′, —CONR′R″, —OC(O)NR′R″, —NR″C(O)R′, —NR′—C(O)NR″R′″, —NR′—SO₂NR′″, —NR″CO₂R′, —NH—C(NH₂)═NH, —NR′C(NH₂)═NH, —NH—C(NH₂)═NR′, —S(O)R′, —SO₂R′, —SO₂NR′R″, —NR″SO₂R, —CN and —NO₂, —N₃, —CH(Ph)₂, perfluoro(C1-C4)alkoxy and perfluoro(C1-C4)alkyl, in a number ranging from zero to three, with those groups having zero, one or two substituents being particularly preferred. R′, R″ and R′″ each independently refer to hydrogen, unsubstituted (C1-C8)alkyl and heteroalkyl, unsubstituted aryl, aryl substituted with one to three halogens, unsubstituted alkyl, alkoxy or thioalkoxy groups, or aryl-(C1-C4)alkyl groups. When R′ and R″ are attached to the same nitrogen atom, they can be combined with the nitrogen atom to form a 5-, 6- or 7-membered ring. Hence, —NR′R″ includes 1-pyrrolidinyl and 4-morpholinyl, “alkyl” includes groups such as trihaloalkyl (e.g., —CF₃ and —CH₂CF₃), and when the aryl group is 1,2,3,4-tetrahydronaphthalene, it may be substituted with a substituted or unsubstituted (C3-C7)spirocycloalkyl group. The (C3-C7)spirocycloalkyl group may be substituted in the same manner as defined herein for “cycloalkyl”.

Preferred substituents are selected from: halogen, —R′, —OR′, ═O, —NR′R″, —SR′, —SiR′R″R′″, —OC(O)R′, —C(O)R′, —CO₂R′, —CONR′R″, —OC(O)NR′R″, —NR″C(O)R′, —NR″CO₂R′, —NR′—SO₂NR″R′″, —S(O)R′, —SO₂R′, —SO₂NR′R″, —NR″SO₂R, —CN and —NO₂, perfluoro(C1-C4)alkoxy and perfluoro(C1-C4)alkyl, where R′ and R″ are as defined above.

The term “fused ring” herein refers to a polycyclic ring system, e.g., a bicyclic or tricyclic ring system, in which two rings share only two ring atoms and one bond in common. Examples of fused rings may comprise a fused bicyclic cycloalkyl ring such as those having from 7 to 12 ring atoms arranged as a bicyclic ring selected from [4,4], [4,5], [5,5], [5,6] and [6,6] ring systems as mentioned above; a fused bicyclic aryl ring such as 7 to 12 membered bicyclic aryl ring systems as mentioned above, a fused tricyclic aryl ring such as 10 to 15 membered tricyclic aryl ring systems mentioned above; a fused bicyclic heteroaryl ring such as 8- to 12-membered bicyclic heteroaryl rings as mentioned above, a fused tricyclic heteroaryl ring such as 11- to 14-membered tricyclic heteroaryl rings as mentioned above; and a fused bicyclic or tricyclic heterocyclyl ring as mentioned above.

The compounds may contain an asymmetric center and may thus exist as enantiomers. Where the compounds possess two or more asymmetric centers, they may additionally exist as diastereomers. Enantiomers and diastereomers fall within the broader class of stereoisomers. All such possible stereoisomers as substantially pure resolved enantiomers, racemic mixtures thereof, as well as mixtures of diastereomers are intended to be included. All stereoisomers of the compounds and/or pharmaceutically acceptable salts thereof are intended to be included. Unless specifically mentioned otherwise, reference to one isomer applies to any of the possible isomers. Whenever the isomeric composition is unspecified, all possible isomers are included.

The compounds of the invention may also contain unnatural proportions of atomic isotopes at one or more of the atoms that constitute such compounds, such as deuterium, e.g. —CD₃, CD₂H or CDH₂ in place of methyl. For example, the compounds may be radiolabeled with radioactive isotopes, such as for example tritium (³H), iodine-125 (¹²⁵I) or carbon-14 (¹⁴C). All isotopic variations of the compounds of the invention, whether radioactive or not, are intended to be encompassed within the scope of the invention.

The term “substantially pure” means that the target stereoisomer contains no more than 35%, such as no more than 30%, further such as no more than 25%, even further such as no more than 20%, by weight of any other stereoisomer(s). In some embodiments, the term “substantially pure” means that the target stereoisomer contains no more than 10%, for example, no more than 5%, such as no more than 1%, by weight of any other stereoisomer(s).

When compounds contain olefin double bonds, unless specified otherwise, such double bonds are meant to include both E and Z geometric isomers.

Some of the compounds may exist with different points of attachment of hydrogen, referred to as tautomers. For example, compounds including carbonyl —CH₂C(O)— groups (keto forms) may undergo tautomerism to form hydroxyl —CH═C(OH)— groups (enol forms). Both keto and enol forms, individually as well as mixtures thereof, are also intended to be included where applicable.

It may be advantageous to separate reaction products from one another and/or from starting materials. The desired products of each step or series of steps is separated and/or purified (hereinafter separated) to the desired degree of homogeneity by the techniques common in the art. Typically such separations involve multiphase extraction, crystallization from a solvent or solvent mixture, distillation, sublimation, or chromatography. Chromatography can involve any number of methods including, for example: reverse-phase and normal phase; size exclusion; ion exchange; high, medium and low pressure liquid chromatography methods and apparatus; small scale analytical; simulated moving bed (“SMB”) and preparative thin or thick layer chromatography, as well as techniques of small scale thin layer and flash chromatography. One skilled in the art will apply techniques most likely to achieve the desired separation.

Diastereomeric mixtures can be separated into their individual diastereomers on the basis of their physical chemical differences by methods well known to those skilled in the art, such as by chromatography and/or fractional crystallization. Enantiomers can be separated by converting the enantiomeric mixture into a diastereomeric mixture by reaction with an appropriate optically active compound (e.g., chiral auxiliary such as a chiral alcohol or Mosher's acid chloride), separating the diastereomers and converting (e.g., hydrolyzing) the individual diastereoisomers to the corresponding pure enantiomers. Enantiomers can also be separated by use of a chiral HPLC column.

A single stereoisomer, e.g., a substantially pure enantiomer, may be obtained by resolution of the racemic mixture using a method such as formation of diastereomers using optically active resolving agents. Racemic mixtures of chiral compounds of the invention can be separated and isolated by any suitable method, including: (1) formation of ionic, diastereomeric salts with chiral compounds and separation by fractional crystallization or other methods, (2) formation of diastereomeric compounds with chiral derivatizing reagents, separation of the diastereomers, and conversion to the pure stereoisomers, and (3) separation of the substantially pure or enriched stereoisomers directly under chiral conditions.

“Pharmaceutically acceptable salts” include, but are not limited to salts with inorganic acids, selected, for example, from hydrochlorates, phosphates, diphosphates, hydrobromates, sulfates, sulfinates, and nitrates; as well as salts with organic acids, selected, for example, from malates, maleates, fumarates, tartrates, succinates, citrates, lactates, methanesulfonates, p-toluenesulfonates, 2-hydroxyethylsulfonates, benzoates, salicylates, stearates, alkanoates such as acetate, and salts with HOOC—(CH₂)n-COOH, wherein n is selected from 0 to 4. Similarly, examples of pharmaceutically acceptable cations include, but are not limited to, sodium, potassium, calcium, aluminum, lithium, and ammonium.

In addition, if a compound is obtained as an acid addition salt, the free base can be obtained by basifying a solution of the acid salt. Conversely, if the product is a free base, an addition salt, such as a pharmaceutically acceptable addition salt, may be produced by dissolving the free base in a suitable organic solvent and treating the solution with an acid, in accordance with conventional procedures for preparing acid addition salts from base compounds. Those skilled in the art will recognize various synthetic methodologies that may be used without undue experimentation to prepare non-toxic pharmaceutically acceptable addition salts.

“Treating,” “treat,” or “treatment” refers to administering at least one compound and/or at least one stereoisomer thereof, and/or at least one pharmaceutically acceptable salt thereof to a subject in recognized need thereof.

An “effective amount” refers to an amount of at least one compound and/or at least one stereoisomer thereof, and/or at least one pharmaceutically acceptable salt thereof effective to “treat” a disease or disorder in a subject, and that will elicit, to some significant extent, the biological or medical response of a tissue, system, animal or human that is being sought, such as when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The term “at least one substituent” includes, for example, from 1 to 4, such as from 1 to 3, further as 1 or 2, substituents. For example, “at least one substituent R” herein includes from 1 to 4, such as from 1 to 3, further as 1 or 2, substituents selected from the list of R as described herein.

The subject compounds and stereoisomers thereof, and pharmaceutically acceptable salts thereof may be employed alone or in combination with at least one other therapeutic agent for treatment. In some embodiments, the compounds, stereoisomers thereof, and pharmaceutically acceptable salts thereof can be used in combination with at least one additional therapeutic agent. The compound and/or one pharmaceutically acceptable salt disclosed herein may be administered with the at least one other therapeutic agent in a single dosage form or as a separate dosage form. When administered as a separate dosage form, the at least one other therapeutic agent may be administered prior to, at the same time as, or following administration of the compound and/or one pharmaceutically acceptable salt disclosed herein.

Also provided is a composition comprising a subject compound and stereoisomers thereof, and pharmaceutically acceptable salts thereof, and at least one pharmaceutically acceptable carrier.

The composition comprising a subject compound and stereoisomers thereof, and pharmaceutically acceptable salts thereof can be administered in various known manners, such as orally, topically, rectally, parenterally, by inhalation spray, or via an implanted reservoir, although the most suitable route in any given case will depend on the particular host, and nature and severity of the conditions for which the active ingredient is being administered. The term “parenteral” as used herein includes subcutaneous, intracutaneous, intravenous, intramuscular, intraarticular, intraarterial, intrasynovial, intrasternal, intrathecal, intralesional and intracranial injection or infusion techniques. The compositions disclosed herein may be conveniently presented in unit dosage form and prepared by any of the methods well known in the art.

The subject compounds and stereoisomers thereof, and pharmaceutically acceptable salts thereof can be administered orally in solid dosage forms, such as capsules, tablets, troches, dragées, granules and powders, or in liquid dosage forms, such as elixirs, syrups, emulsions, dispersions, and suspensions. The subject compounds and stereoisomers thereof, and pharmaceutically acceptable salts thereof disclosed herein can also be administered parenterally, in sterile liquid dosage forms, such as dispersions, suspensions or solutions. Other dosages forms that can also be used to administer the subject compounds and stereoisomers thereof, and pharmaceutically acceptable salts thereof disclosed herein as an ointment, cream, drops, transdermal patch or powder for topical administration, as an ophthalmic solution or suspension formation, i.e., eye drops, for ocular administration, as an aerosol spray or powder composition for inhalation or intranasal administration, or as a cream, ointment, spray or suppository for rectal or vaginal administration.

Gelatin capsules containing the compound and/or the at least one pharmaceutically acceptable salt thereof disclosed herein and powdered carriers, such as lactose, starch, cellulose derivatives, magnesium stearate, stearic acid, and the like, can also be used. Similar diluents can be used to make compressed tablets. Both tablets and capsules can be manufactured as sustained release products to provide for continuous release of medication over a period of time. Compressed tablets can be sugar coated or film coated to mask any unpleasant taste and protect the tablet from the atmosphere, or enteric coated for selective disintegration in the gastrointestinal tract.

Liquid dosage forms for oral administration can further comprise at least one agent selected from coloring and flavoring agents to increase patient acceptance.

In general, water, a suitable oil, saline, aqueous dextrose (glucose), and related sugar solutions and glycols such as propylene glycol or polyethylene glycols can be examples of suitable carriers for parenteral solutions. Solutions for parenteral administration may comprise a water soluble salt of the at least one compound describe herein, at least one suitable stabilizing agent, and if necessary, at least one buffer substance. Antioxidizing agents such as sodium bisulfite, sodium sulfite, or ascorbic acid, either alone or combined, can be examples of suitable stabilizing agents. Citric acid and its salts and sodium EDTA can also be used as examples of suitable stabilizing agents. In addition, parenteral solutions can further comprise at least one preservative, selected, for example, from benzalkonium chloride, methyl- and propylparaben, and chlorobutanol.

A pharmaceutically acceptable carrier is, for example, selected from carriers that are compatible with active ingredients of the composition (and in some embodiments, capable of stabilizing the active ingredients) and not deleterious to the subject to be treated. For example, solubilizing agents, such as cyclodextrins (which can form specific, more soluble complexes with the at least one compound and/or at least one pharmaceutically acceptable salt disclosed herein), can be utilized as pharmaceutical excipients for delivery of the active ingredients. Examples of other carriers include colloidal silicon dioxide, magnesium stearate, cellulose, sodium lauryl sulfate, and pigments such as D&C Yellow #10. Suitable pharmaceutically acceptable carriers are described in Remington's Pharmaceutical Sciences, A. Osol, and other reference texts in the art.

For administration by inhalation, the subject compounds and stereoisomers thereof, and pharmaceutically acceptable salts thereof may be conveniently delivered in the form of an aerosol spray presentation from pressurized packs or nebulisers. The subject compounds and stereoisomers thereof, and pharmaceutically acceptable salts thereof may also be delivered as powders, which may be formulated and the powder composition may be inhaled with the aid of an insufflation powder inhaler device. One exemplary delivery system for inhalation can be metered dose inhalation (MDI) aerosol, which may be formulated as a suspension or solution of a subject compound and stereoisomers thereof, and pharmaceutically acceptable salts thereof disclosed herein in at least one suitable propellant, selected, for example, from fluorocarbons and hydrocarbons.

For ocular administration, an ophthalmic preparation may be formulated with an appropriate weight percentage of a solution or suspension of the subject compound and stereoisomers thereof, and pharmaceutically acceptable salts thereof in an appropriate ophthalmic vehicle, such that the subject compound and stereoisomers thereof, and at least one pharmaceutically acceptable salts thereof is maintained in contact with the ocular surface for a sufficient time period to allow the compound to penetrate the corneal and internal regions of the eye.

Useful pharmaceutical dosage-forms for administration of the subject compounds and stereoisomers thereof, and pharmaceutically acceptable salts thereof disclosed herein include, but are not limited to, hard and soft gelatin capsules, tablets, parenteral injectables, and oral suspensions.

The dosage administered will be dependent on factors, such as the age, health and weight of the recipient, the extent of disease, type of concurrent treatment, if any, frequency of treatment, and the nature of the effect desired. In general, a daily dosage of the active ingredient can vary, for example, from 0.1 to 2000 milligrams per day. For example, 10-500 milligrams once or multiple times per day may be effective to obtain the desired results.

In some embodiments, a large number of unit capsules can be prepared by filling standard two-piece hard gelatin capsules each with, for example, 100 milligrams of the subject compound and stereoisomers thereof, and pharmaceutically acceptable salt thereof disclosed herein in powder, 150 milligrams of lactose, 50 milligrams of cellulose, and 6 milligrams magnesium stearate.

In some embodiments, a mixture of the compound, stereoisomers thereof, and pharmaceutically acceptable salts thereof a digestible oil such as soybean oil, cottonseed oil or olive oil can be prepared and injected by means of a positive displacement pump into gelatin to form soft gelatin capsules containing 100 milligrams of the active ingredient. The capsules are washed and dried.

In some embodiments, a large number of tablets can be prepared by conventional procedures so that the dosage unit comprises, for example, 100 milligrams of the compound, stereoisomers thereof, and pharmaceutically acceptable salts thereof, 0.2 milligrams of colloidal silicon dioxide, 5 milligrams of magnesium stearate, 275 milligrams of microcrystalline cellulose, 11 milligrams of starch and 98.8 milligrams of lactose. Appropriate coatings may be applied to increase palatability or delay absorption.

In some embodiments, a parenteral composition suitable for administration by injection can be prepared by stirring 1.5% by weight of the compound and/or at least an enantiomer, a diastereomer, or pharmaceutically acceptable salt thereof disclosed herein in 10% by volume propylene glycol. The solution is made to the expected volume with water for injection and sterilized.

In some embodiment, an aqueous suspension can be prepared for oral administration. For example, each 5 milliliters of an aqueous suspension comprising 100 milligrams of finely divided compound, stereoisomers thereof, and pharmaceutically acceptable salts thereof, 100 milligrams of sodium carboxymethyl cellulose, 5 milligrams of sodium benzoate, 1.0 grams of sorbitol solution, U.S.P., and 0.025 milliliters of vanillin can be used.

The same dosage forms can generally be used when the compound, stereoisomers thereof, and pharmaceutically acceptable salts thereof are administered stepwise or in conjunction with at least one other therapeutic agent. When drugs are administered in physical combination, the dosage form and administration route should be selected depending on the compatibility of the combined drugs. Thus the term coadministration is understood to include the administration of at least two agents concomitantly or sequentially, or alternatively as a fixed dose combination of the at least two active components.

The compounds, stereoisomers thereof, and pharmaceutically acceptable salt thereof disclosed herein can be administered as the sole active ingredient or in combination with at least one second active ingredient.

The subject compounds are incorporated into pharmaceutical compositions or formulations. The compositions will contain pharmaceutically acceptable diluents and/or carriers, i. e. diluents or carriers that are physiologically compatible and substantially free from pathogenic impurities. Suitable excipients or carriers and methods for preparing administrable compositions are known or apparent to those skilled in the art and are described in more detail in such publications as Remington's Pharmaceutical Science, Mack Publishing Co, NJ (1991). The compositions may also be in the form of controlled release or sustained release compositions as known in the art. For many applications the subject compounds are administered for morning/daytime dosing, with off period at night.

The subject compounds may be used per se, or in the form of their pharmaceutically acceptable salts, such as hydrochlorides, hydrobromides, acetates, sulfates, citrates, carbonates, trifluoroacetates and the like. When compounds contain relatively acidic functionalities, salts can be obtained by addition of the desired base, either neat or in a suitable inert solvent. Examples of pharmaceutically acceptable base addition salts include sodium, potassium, calcium, ammonium, organic amino, or magnesium salts, or the like. When compounds contain relatively basic functionalities, salts can be obtained by addition of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, maleic, malonic, benzoic, succinic, suberic, fumaric, lactic, mandelic, phthalic, benzenesulfonic, p-tolylsulfonic, citric, tartaric, methanesulfonic, and the like. Also included are salts of amino acids such as arginate and the like, and salts of organic acids like glucuronic or galacturonic acids and the like.

The neutral forms of the compounds may be regenerated by contacting the salt with a base or acid, and isolating the parent compound in the conventional manner. The parent form of the compound differs from the various salt forms in certain physical properties, such as solubility in polar solvents, but otherwise the salts are equivalent to the parent form of the compound for the purposes of this invention.

In addition to salt forms, this invention provides compounds which are in a prodrug form. Prodrugs of the compounds described herein are those compounds that readily undergo chemical changes under physiological conditions to provide the compounds of the present invention. Additionally, prodrugs can be converted to the compounds of the present invention by chemical or biochemical methods in an ex vivo environment. For example, prodrugs can be slowly converted to the compounds of the present invention when placed in a transdermal patch reservoir with a suitable enzyme or chemical reagent. Prodrugs are often useful because, in some situations, they may be easier to administer than the parent drug. They may, for instance, be more bioavailable by oral administration than the parent drug. The prodrug may also have improved solubility in pharmacological compositions over the parent drug. A wide variety of prodrug derivatives are known in the art, such as those that rely on hydrolytic cleavage or oxidative activation of the prodrug. An example, without limitation, of a prodrug would be a compound of the present invention which is administered as an ester (the “prodrug”), but then is metabolically hydrolyzed to the carboxylic acid, the active entity.

Certain compounds of the invention can exist in unsolvated forms as well as solvated forms, including hydrated forms. In general, the solvated forms are equivalent to unsolvated forms and are intended to be encompassed within the scope of the present invention. Certain compounds of the invention may exist in multiple crystalline or amorphous forms. In general, all physical forms are equivalent for the uses contemplated by the present invention and are intended to be within the scope of the invention.

Some of the subject compounds possess asymmetric carbon atoms (optical centers) or double bonds; the racemates, diastereomers, geometric isomers and individual isomers are all intended to be encompassed within the scope of the present invention.

The compounds are generally administered in a “therapeutically effective amount”, i.e. the amount of the subject compound that will elicit the biological or medical response of a tissue, system, animal or human that is being sought by the researcher, veterinarian, medical doctor or other clinician. The term “therapeutically effective amount” includes that amount of a compound that, when administered, is sufficient to prevent development of, or alleviate to some extent, one or more of the symptoms of the condition or disorder being treated. The therapeutically effective amount will vary depending on the compound, the disease and its severity and the age, weight, etc., of the mammal to be treated.

The contacting is generally effected by administering to the subject an effective amount of one or more compounds having a formula herein, including the various embodiments described above. Generally administration is adjusted to achieve a therapeutic dosage of about 0.1 to 50, preferably 0.5 to 10, more preferably 1 to 10 mg/kg, though optimal dosages are compound specific, and generally empirically determined for each compound.

The term “unit dosage forms” refers to physically discrete units suitable as unitary dosages for human subjects and other mammals, each unit containing a predetermined quantity of active material calculated to produce the desired therapeutic effect, in association with a suitable pharmaceutical excipient. Typical unit dosage forms include prefilled, premeasured ampules or syringes of the liquid compositions or pills, tablets, capsules, lozenges or the like in the case of solid compositions. In such compositions, the mimetic is usually a minor component (from about 0.1 to about 50% by weight or preferably from about 1 to about 40% by weight) with the remainder being various vehicles or carriers and processing aids helpful for forming the desired dosing form. Unit dosage formulations are preferably about of 5, 10, 25, 50, 100, 250, 500, or 1,000 mg per unit. In a particular embodiment, unit dosage forms are packaged in a multipack adapted for sequential use, such as blisterpack comprising sheets of at least 6, 9 or 12 unit dosage forms.

### Example: Thiophenyl Derivatives of Nicotinamide are Metabolized by the NAD Salvage Pathway into Unnatural NAD Derivatives that Inhibit IMPDH and are Toxic to Neuronal Cancers

The N-pyridinylthiophene carboxamide, compound 21 (1), has a simple chemical structure composed of pyridine and thiophene rings connected through a carboxamide linkage and is cytotoxic to the human MPNST cell line S462 with a half-maximal inhibitory concentration (IC50) of 1.41 μM. We synthesized 66 analogs of 1 in a medicinal chemistry effort to assess the importance of these chemical groups to the cytotoxic activity in S462 cells. Strikingly, all chemical modifications to the pyridinyl group made the compound inactive up to 50 μM (IC50>50 μM), the highest dose tested. These modifications included replacement of the pyridinyl group with a phenyl (2), moving the 3-pyridinyl nitrogen to all possible alternate positions (2- and 4-pyridinyls 3 and 4), and addition of a methyl group at all possible positions within the pyridinyl ring (analogs 5-8). Taken together, these observations suggest that a 3-pyridinyl group is essential to the activity of 1. We also found that replacement of the amide with an ester, or alkylation of the amide nitrogen abolished potency (IC50s>50 μM), while the thiophene ring was more permissive to modifications (see appendix below: Synthesis, characterization and activity of micromolar and nanomolar active nicotinamide derivatives). On the other hand, reversal of the carboxamide, leading to the N-thiophenylnicotinamide analog 9, led to a slightly increased potency (IC50 of 1.20 μM).

We noted that 9, the most potent derivative identified, contained within its structure the vitamin nicotinamide (Nam). This similarity, in addition to the scaffold's particular sensitivity to modifications around the pyridinyl ring led us to hypothesize that the activity of compounds 1 and 9 may depend on the concentration of Nam in the media. To test this hypothesis, we analyzed the activity of both compounds in media supplemented with increasing concentrations of Nam. The IC50 of 1 increased from 1.96 μM to 6.85 and 32.3 μM with the addition of 100 μM and 1 mM exogenous Nam, respectively. These respective concentrations of Nam also led to a comparable rescue in the toxicity of 9 for which the potency shifted from 0.845 μM to 4.23 μM and 35.7 μM. Given that Nam inhibited compound activity, we considered Nam concentrations in basal culture conditions (DMEM supplemented with 10% fetal bovine serum or FBS) as compared to physiological levels. Nam levels in human serum are estimated to be in the 4 μM range.⁵ In contrast, DMEM alone contains 33 M of Nam and the concentration in 10% FBS is expected to range between 0.574-1.56 μM.⁶ To more closely reflect physiologic levels of Nam, we evaluated the activity of these compounds in cells grown in DMEM lacking Nam (Nam-free DMEM), so that the only exogenous Nam derives from 10% FBS. Importantly, culturing cells in Nam-free DMEM with 10% FBS did not affect cell viability over multiple passages. When cells were cultured in Nam-free DMEM, the potency of analog 9 in S462 cells increased 92-fold (IC50=9.17 nM). Moreover, the IC50 of 9 progressively increased to 51.4 nM and 1.84 μM with the addition of 1.4 μM and 37 μM Nam, respectively. These observations indicate that the potency of these compounds is in the nanomolar range when tested in the presence of physiological levels of Nam.

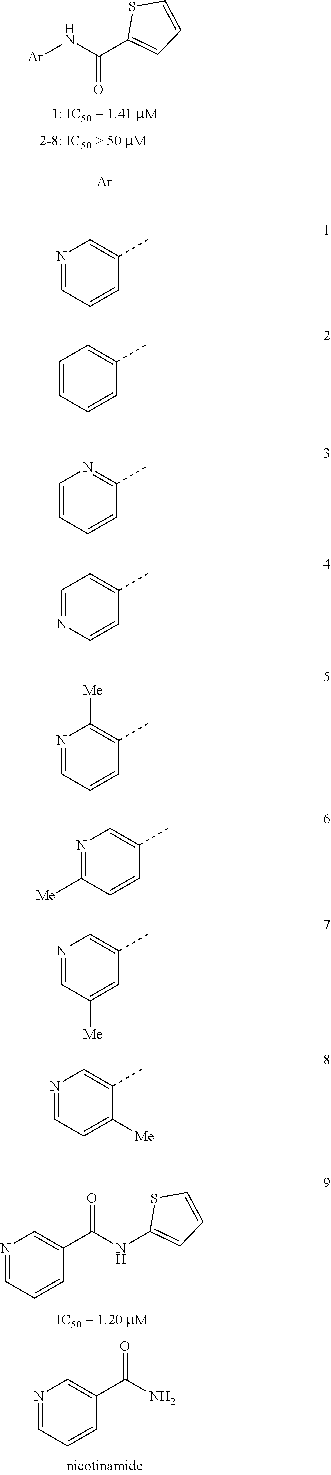

Compound 21 analogs and IC50s in S462 (MPNST) cells in commercial DMEM. The structure of the optimized compound 21 analog, 9, in comparison to the vitamin, nicotinamide (Nam).

We thus re-assessed the structure activity relationship of compound 21 analogs in Nam-free DMEM supplemented with or without 100 μM Nam (see appendix below: Synthesis, characterization and activity of micromolar and nanomolar active nicotinamide derivatives). Without exogenous Nam, 18 out of 67 compounds, which previously were found to have no cytotoxic activity, were now cytotoxic with an IC50 less than 1 μM. Moreover, all of these active compounds were rescued by the addition of 100 μM Nam to the media demonstrating that their activity, like compound 21, is dependent on Nam levels. Particularly notable, even in Nam-depleted conditions, all active analogs retained the same 3-pyridinyl ring as Nam, reinforcing the relationship between structural resemblance to Nam and compound efficacy.

The ability of Nam to rescue the toxicity of compound 21 derivatives suggests that the activity of these compounds affects essential pathways involving Nam. In the NAD salvage pathway, Nam is converted by nicotinamide phosphoribosyltransferase (NAMPT) into nicotinamide mononucleotide (NMN), which is then converted into nicotinamide NAD by one of three different nicotinamide mononucleotide adenylyltransferase (NMNAT) enzymes including NMNAT1, NMNAT2, and NMNAT3.⁷ NAMPT inhibitors like FK866 are known to cause cancer cell death, and thus we considered that 9 may competitively inhibit NAMPT, a hypothesis that might explain the ability of Nam to rescue toxicity.^{8,9} We first tested whether 1 or 9 inhibits the conversion of Nam to NAD in a previously reported cell-free coupled enzyme assay.¹⁰ This assay quantifies NADH production from Nam in the presence of phosphoribosyl pyrophosphate (PRPP), ATP, NAMPT, NMNAT1 and alcohol dehydrogenase, the last of which is necessary to reduce NAD to the fluorescent NADH. The addition of either 1 or 9 to this enzyme reaction did not appreciably inhibit NAD synthesis (IC50=27 μM for 1, IC50=48 μM for 9) in comparison to FK866 (IC50=0.36 μM). To directly test whether 9 inhibits NAMPT activity in cells, we measured the levels of NMN in S462 cells in regular DMEM following treatment with either 9 or FK866. Increasing concentrations of 9 did not reduce the levels of NMN to the same extent as FK866, supporting the hypothesis that 9 has a mechanism of action distinct from that of NAMPT inhibitors. Taken together, using both cell-based and cell-free experiments, 1 and 9 were toxic to cells via a mechanism other than inhibition of canonical NAD salvage.

To gain insight into the mechanism of 9 and the relevance of Nam, we pursued two independent and complementary unbiased genetic approaches. First, we performed a genome-wide pooled Clustered Regularly Interspaced Short Palindromic Repeats (CRISPR) and CRISPR-associated protein 9 (Cas9) knockout screen to identify genes whose loss of function can enhance or suppress compound 9 activity. We found that 9 was toxic to Cas9-expressing HCT116 when cultured in 10% FBS Nam-free DMEM (IC50=20.8 nM), and this activity was rescued by 100 μM Nam, indicating the same mechanism that was observed in S462 cells. We cultured Cas9-expressing HCT116 cells in 10% FBS Nam-free DMEM and transduced with a lentiviral single guide RNA (sgRNA) library designed to silence 19,114 human genes.¹¹ These cells were treated with either vehicle (DMSO) or 9 for 30 days by which time the 9-treated cells had 7.6 fewer population doublings. Then, relative sgRNA sequences in the resulting cells were determined by massively parallel sequencing of PCR products amplified from genomic DNA, and the relative impact of each gene was derived from the sgRNA reads.

The largest difference was a 5.2-fold increase in the sgRNA reads (evaluated using the MAGeCK algorithm targeting NMNAT1 in 9-treated cells. The sgRNA reads for the NAMPT gene were enriched by 2.1-fold in 9-treated cells, making it the 101^{st} most enriched gene out of 19,114. To test whether a decrease in NMNAT 1 protein influences the activity of 9, we used CRISPR/Cas9 to knock out NMNAT1 using three independent sgRNAs in both HCT116 and S462 cells. Guide RNAs led to a reduction in NMNAT1 protein and concomitantly increased the IC50 of 9 in S462 and HCT116 cells by an average of 28.5- and 6.0-fold, respectively. To explore the role of NAMPT, we added sublethal concentrations of FK866 to varying doses of 9. The addition of 278 pM and 833 pM of FK866 led to resistance to 9 by an increase in the IC50 by 2.9- and 15.4-fold, respectively. These observations, taken together, support the hypothesis that the activity of compound 9 depends on NAMPT and NMNAT1.

Second, we screened for resistance to 9 in iHCT116 cells, a system that uses inducible mutagenesis and forward genetics to find compound-resistant mutations and thus can reveal both loss of function and gain of function alleles that suppress activity. The iHCT116 system is a barcoded cell line in which we temporally control mutagenesis by the addition of indole acetic acid (IAA) mediated degradation of the mismatch repair protein MLH1.¹² MLN4924 is a small molecule toxin that we use as a positive control to verify that IAA treatment leads to increased mutations. By degrading MLH1 and increasing the overall mutation rate, IAA treatment increases the frequency of UBA3 mutations, leading to a higher rate of MLN4924 resistance. Consistent with prior results, following selection with MLN4924, 33 resistant clones were identified in the IAA treated (or Mut-on) condition compared to only one clone in the vehicle (or Mut-off) condition, demonstrating that IAA induced mutagenesis.¹² We then performed selections with increasing lethal doses of 9 in Mut-off or Mut-on population of cells. Although we were able to isolate resistant clones at all three selection concentrations of 9, we did not observe a substantial difference in the number of surviving clones between the Mut-off or Mut-on populations. A comparable rate of clonal resistance between Mut-on and Mut-off conditions suggests that resistance may be influenced by gene expression through a mechanism other than mutation. Having previously established a relationship between either NMNAT1 levels or NAMPT activity and compound resistance, we analyzed the levels of both enzymes in multiple resistant clones that harbored unique barcode sequences. Indeed, we found that NMNAT1 protein levels were downregulated in five out of eight clones, whereas NAMPT levels were the same in all clones. Notably, no mutation was identified in either NAMPT or NMNATs in any of these clones using whole exome sequencing, given adequate read depth in these genes. To test whether reduced NMNAT1 levels in these clones contributed to resistance, we ectopically expressed NMNAT1 in either the parental cells or a representative resistant clone with low NMNAT1 expression. Ectopic expression of NMNAT1 restored sensitivity of the resistant clone to 9 by a 9.58-fold decrease in IC50 from 0.574 μM to 59.9 nM. Ectopic expression of NMNAT1 in parental iHCT116 cells also decreased the IC50 to 9 by 1.85-fold (mock-infected IC50=49.7 nM to NMNAT1-expression IC50=26.9 nM). Altogether, these findings suggest that the levels of NMNAT1 determine sensitivity to 9 and that a subset of 9-resistant iHCT116 clones are due to epigenetic changes that reduce the expression of NMNAT1. In summary, two independent unbiased strategies for understanding compound mechanism both demonstrate that enzymes involved in NAD synthesis are also necessary for compound 9 toxicity.

Given that compound 21 (1) and 9 both resemble and are rescued by Nam, and the fact that the NAD salvage pathway is essential for toxicity, we considered that these two compounds may themselves be substrates in the NAD salvage pathway. In this model, we would predict that the compounds would be metabolized in cells, and that excess Nam acting as a competitive substrate would block that metabolism. To test this hypothesis, we measured the total amount of 9 in media and cells over time with or without Nam supplementation. We found that the levels of 9 in 10% FBS Nam-free DMEM decrease with a half-life of approximately 3.8 hours. By contrast, there was no appreciable decrease in the levels of 9 after 24 hours if the cells were supplemented with 100 μM Nam. These observations suggest that Nam rescues the toxicity of 9 by acting as a competitive substrate for enzymes that metabolize 9.

We reasoned that 1 and 9 would likely be metabolized to 1-mononucleotide (1-MN) and 9-mononucleotide (9-MN) by NAMPT and then to 1-adenine dinucleotide (1-AD) and 9-adenine dinucleotide (9-AD) by NMNAT1. Thus, we analyzed extracts from cell-free reactions that co-incubate each compound with NAMPT, NMNAT1, PRPP, and ATP by high-resolution mass spectrometry to identify these putative metabolites based on exact mass. We observed a time-dependent decrease in the levels of both parent compounds and a corresponding increase in the appearance of the predicted MN and AD species. Of note, 9-MN levels peaked faster and were consumed after 60 minutes, while 1-MN levels were still increasing at 60 minutes. Furthermore, 9-AD also appeared earlier than 1-AD. To test whether these compound metabolites were also produced in cells, we analyzed metabolic extracts from cells treated with either 1 or 9. Similar to the experiment performed in vitro, we observed time-dependent depletion of the parent compounds and accumulation of the compound-derived metabolites in S462 cells, again noting that 9-AD accumulated faster than 1-AD. These observations suggest that 9 and 9-MN, which are more structurally related to Nam and NMN than 1 or 1-MN, may be superior substrates for NAMPT and NMNAT1, respectively. This difference may also explain the slightly improved anti-proliferative potency of 9 relative to 1.

It has already been established that compound 21 is bioavailable in mice and reduces the growth of xenograft tumors.⁴ Thus, we tested whether the corresponding MN and AD metabolites could also be detected in mice following compound administration. GBM, which is reported to be a potential target for these compounds, is an intracranial tumor, and thus, effective drugs must cross the blood-brain barrier. Therefore, in addition to plasma, we analyzed compound and metabolite levels in mouse brain. Following the administration of a single 5 mg/kg intravenous (IV) injection of either 1 or 9, we quantified the levels of each compound in both plasma and brain. In plasma, 1 and 9 levels reached a respective maximum concentration (Cₘₐₓ) of 1155 ng/ml (5.65 μM) and 4833 ng/ml (23.7 μM), and both compounds were rapidly cleared by 90 minutes. The respective brain Cₘₐₓ for 1 and 9 was 2215 ng/g and 1600 ng/g, which converts to 10.8 μM and 7.8 μM assuming a brain density of 1 g/ml. Both compounds persisted longer in the brain than in the plasma which was reflected in increased exposure. The brain to blood exposure, represented by the ratio of the area under the curve between brain and plasma (AUC_{Brain}/AUCₚₗₐₛₘₐ), was 3.45 for 1 and 2.85 for 9.

Next, we analyzed the levels of metabolites that result from the metabolism of 1 and 9 through the NAD salvage pathway. 1-MN and 9-MN reached a peak level in plasma of 1.13×10⁵ and 1.19×10⁵ analyte/internal standard peak area, respectively, which, for comparison, is on the same order of magnitude as what we detected in cells. 1-AD and 9-AD reached a peak of 4.34×10⁴ and 7.55×10⁴ analyte/internal standard peak area, respectively, which was also a similar order of magnitude to our cell-based experiments. However, in comparison to plasma, 1-MN, 1-AD, 9-MN and 9-AD were not reproducibly detected in replicate brain samples, suggesting that they are either not present or present at trace levels. Overall, this data suggests that 1 and its more potent analog 9 are both brain-penetrant, and they are both metabolized in mouse plasma to their respective MN and AD metabolites. In addition, the apparent absence of MN and AD metabolites in brain when compared to cancer cells suggests differences in the NAD salvage pathway between normal brain and neuronal cancers.

The experiments described above are consistent with the model that both 1 and 9 are converted by NAMPT and NMNAT1 into their respective MN and AD metabolites. It remains unclear, however, which of these two metabolites is contributing to toxicity. To address this question, we measured 9 metabolism, 9-MN and 9-AD levels upon treatment of the resistant clones identified through forward genetics. We found that 9 is metabolized over time, and 9-MN levels were similar between the parental cell line and each of these clones. On the other hand, 9-AD levels were reduced in all resistant clones. Together, these results suggest that 9-AD rather than 9-MN is the toxic metabolite. Clones 5 and 7 were resistant to 9 but had normal NAMPT and NMINAT levels. In these two clones, both the levels of 9-MN and 9-AD were reduced, suggesting that perhaps 9 may have been metabolized through an alternative pathway into an inert metabolite. Clone 6 had increased levels of 9-MN but not 9-AD, suggesting that NMNAT1 activity may be inhibited through an alternative mechanism. Overall, this data shows that resistance to 9 is the consequence of failed metabolism into 9-AD and provides evidence that 9-AD, rather than 9-MN, is the toxic metabolite.

Three other small molecules, 3-acetylpyridine (3-AP), vacor and tiazofurin, depend on the NAD salvage pathway for their activation into cytotoxic nucleotide derivatives.¹³⁻¹⁷ Specifically, vacor and 3-AP are activated by NAMPT and NMNAT into vacor-AD and 3-AP-AD derivatives, while tiazofurin is activated by nicotinate riboside kinase (NRK) and NMNAT into thiazole-4-carboxamide adenine dinucleotide (TAD). TAD kills cells through inhibition of IMPDH, an essential enzyme of guanylate synthesis. On the other hand, vacor and 3-AP induce axon degeneration via activation of the NAD hydrolase Sterile Alpha and Toll Interleukin Receptor Motif containing protein 1 (SARM1). They are also known to induce cancer cell death, although the role of SARM1 for the anti-cancer activity is unknown. To investigate the mechanism of compound 21 toxicity downstream of compound toxification, we considered that it might induce cancer cell death through a mechanism similar to one of these compounds. Thus, we compared the cytotoxicity of vacor, 3-AP, tiazofurin and 9 in 10% FBS Nam-free DMEM media. We found that all compounds were toxic to S462, however, 9 was dramatically more potent; its IC50 was 206-fold less than vacor, 1823-fold less than 3-AP and 477-fold less than tiazofurin. Consistent with their known activation mechanisms by the NAD salvage pathway where vacor and 3-AP are dependent on NAMPT and NMNAT, while tiazofurin is dependent on NRK and NMNAT, all compounds except tiazofurin were rescued by nicotinamide.

Given that vacor, 3-AP, and compound 21 are all metabolized to toxic, NAD-like derivatives through NAMPT and NMNAT, we hypothesized that their anti-cancer activity may also depend on SARM1. To test this, we compared the toxic effects of vacor, 3-AP, and 9 in either wild type cells or cells in which we used CRISPR to silence SARM1. SARM1-knockout cells were equally sensitive to 3-AP and 9 but were 3-fold more resistant to vacor. These results demonstrate that SARM1 is necessary for cytotoxicity mediated by vacor, but is not necessary for the cytotoxicity of 3-AP or 9. These observations highlight that even though these different scaffolds are toxified through the same mechanism, the resulting metabolites may have different activities. We next co-titrated guanosine, which is known to rescue tiazofurin by bypassing IMPDH in guanylate synthesis, with each of the above small molecules. We confirmed guanosine rescue of tiazofurin and observed a profound dose-dependent rescue of 9, but no rescue of vacor or 3-AP toxicity. These results suggested that 9-AD is an IMPDH inhibitor. To test this hypothesis, we set up in vitro reactions with different combinations of 9, NAMPT and NMNAT. After incubation, metabolites were extracted, reconstituted in aqueous solution and then co-incubated with an established IMPDH2 enzyme assay. Only extracts from reactions that included 9, NAMPT, and NMNAT, which are known to generate 9-AD, showed substantial inhibition of IMPDH activity. Taken together, these data indicate that the toxicity of 9 results from its conversion into 9-AD, which inhibits IMPDH.

### Methods

### Cell Viability Assay

In 96-well assay plates (Corning #2903), 2,000 cells were plated per well in 100 μL of medium and allowed to adhere overnight. The next day, compounds or Nam suspended in DMSO were dispensed to the assayed wells using a D300e Digital Dispenser (Tecan). All wells were normalized to 0.5% DMSO. 72 hours after incubation at 37 °C in 5% CO₂, cell viability was assayed using an ATP-based luminescent viability assay, CellTiter-Glo (Promega #G8462). CellTiter-Glo substrate was mixed 1:1 with phosphate-buffered saline (PBS) supplemented with 1% Triton X-100, and 40 μL of this mixture was added to the assay wells. After the plates were shaken on a microplate shaker for 10 minutes at room temperature, luminescence was recorded using a Synergy 2 Microplate Reader or Cytation 5 plate reader (BioTek). In Prism 9 software (GraphPad), ten-point dose response curves were plotted with baseline-correction to the vehicle control well, and IC50 values were calculated by curve fitting using asymmetric (five parameter), least squares fit.

### Cell-Free Production of 1 and 9 Metabolites

A reaction mixture containing 50 mM Tris pH 8, 0.4 mM PRPP, 2.5 mM ATP, 12 mM MgCl2, 0.02% BSA, 1 μM NAMPT, 100 nM NMNAT1 and 200 μM compound 9 or 1 was incubated at 37 °C. Fifty microliter aliquots were quenched at the indicated time points by adding two volumes of acetonitrile with 0.1% formic acid and vortexing for 15 seconds. After incubation for ten minutes at room temperature, samples were centrifuged at 13,200 rpm for 5 minutes. The supernatants were filtered through 0.2 μm PVDF filters (Sigma #SLGV004SL) and analyzed by LC-MS/TOF.

Production of 1 and 9 Metabolites in S462 and iHCT116

Prior to the experiment, cells were cultured in low-Nam DMEM for at least 1 passage (3 days) to deplete intracellular Nam. 15,000 cells were plated in a 96-well plate with 100 μL low-Nam media. At appropriate time points, 500 nM 1 or 9, or equal volume of vehicle (DMSO) were added using TECAN. 200 μL of acetonitrile with 0.1% formic acid were added to the assayed wells. For the 0-hour time point, compound or vehicle was added after the addition of acetonitrile and formic acid. Following incubation for 10 minutes at room temperature, samples were transferred to Eppendorf tubes and centrifuged at 20,000×g for 5 minutes. The supernatants were filtered through 0.2 μm PVDF filters and analyzed by LC-MS/TOF.

### LC-MS Analysis

Initial evaluation of the stability of 9 in the presence and absence of exogenous Nam was performed using LC-MS/MS analysis with a Sciex TripleQuad™ 4500 (AB SCIEX, Framingham, MA, USA) coupled to a Shimadzu (Columbia, MD) Prominence LC. The compound was detected in positive MRM mode following the transition 204.9 to 79.0. Instrument settings were as follows: Dwell time of 150 ms, DP (declustering potential) 81.0 V, EP (entrance potential) 10.0 V, CE (collision energy) 47 V, CXP (collision cell exit potential) 8 V, CUR (curtain gas) 35 V, CAD (collision gas) med, IS (ion spray voltage) 4500 V, TEM (turbo heater temperature) 700 °C, GS1 (nebulizing gas) 60 psi, GS2 (auxiliary gas) 60 psi. Tolbutamide (Sigma, St. Louis, MO; transition 212.1/91.1) was used as an internal standard and analyte peak areas were normalized to IS peak areas for evaluation of compound levels over time. IS settings were similar to 9 except DP of 36.0 V, CE of 29 V, EP of 4.5 V and CXP of 4.0V. An Agilent C18 XDB column (5 micron, 50×4.6 mm) was used for chromatography with the following conditions: Solvent A: water+0.1% formic acid (LC-MS grade); Solvent B: acetonitrile+0.1% formic acid (LC-MS grade). Gradient conditions were as follows: 0-1.5 min 3% B, 1.5-2.0 min gradient to 100% B, 2.0-3.5 min 100% B, 3.5-3.6 min gradient to 3% B, 3.6-4.5 min 3% B. The column temperature, sample injection volume, and flow rate were set to 36 °C, 5 μl, and 1.5 ml/min, respectively.

Subsequent Mass spectrometric analysis of 1 and 9 and their MN and AD metabolite levels in cell culture were performed using a Sciex TripleTOF® 6600 system equipped with an electrospray ionization (ESI) source used in the positive ionization mode, and configured as follows: Ion Source Gas 1 (Gas 1), 50 psi; Ion Source Gas 2 (Gas 2), 45 psi; curtain gas flow, 25 psi; source temperature, 500 °C; and ion spray voltage floating, +5500 V(+). TOF-MS mode (Full scan) and Information Dependent Acquisition (IDA) mode (Product Ion scan) were utilized to collect MS and MS/MS data, respectively. For TOF-MS scans, the mass range was from m/z 70 to 1000 and for Product Ion scans, the mass range was from m/z 30 to 1000. The collision energy (CE) was set at 30 V (+) collision energy spread (CES) was ±15 V. The accumulation time was 0.25 seconds for TOF-MS scans and 0.06 seconds for product ion scans. The instrument was automatically calibrated using a calibration delivery system injected in APCI positive every 5 samples. The mass spectrometer was coupled to a Shimadzu HPLC (Nexera X2 LC-30AD). The system was controlled by Analyst TF 1.8.1 software (Sciex).

Chromatography was performed under reverse phase conditions using a Luna Omega 3 micron, C18, 100 A, 150×2.1 mm (Phenomenex, Torrance, CA). The column temperature, sample injection volume, and flow rate were set to 30 °C, 2 μL, and 0.2 mL/min, respectively. The HPLC conditions were as follows: Solvent A: water including 0.1% formic acid (LC-MS) grade. Solvent B: Acetonitrile including %0.1 formic acid (LC-MS) grade. Gradient condition was 0-3 min: 0% B, 25 min: 95% B, 29 min: 95% B, 29.5 min: 0% B, 35 min: 0% B for a total run time of 35 mins.

For PK studies, a slightly modified chromatography method using the same Luna Omega column was employed which reduced run time but did not affect peak retention times. MS conditions were identical. The HPLC conditions were as follows: Solvent A: water including 0.1% formic acid (LC-MS) grade. Solvent B: Acetonitrile including %0.1 formic acid (LC-MS) grade. Gradient condition was 0-3 min: 3% B, 17 min: 60.5% B, 17.1 min Flow to 0.25 mL/min, 17 to 20 min hold 95% B: 20.5-25 min hold 3% B, 24.5 flow to 0.2 mL/min. Total run time: 25 mins.

Data analysis for TOF data was performed using SCIEX MultiQuant software version 3.0.3. For each of the compounds monitored, the theoretical exact m/z was calculated. A range of ±10 ppm was extracted around the theoretical mass and used to create an analytical method in MultiQuant. Extracted areas for parent compounds 1 and 9 in S462 cells and mice were normalized to the internal standard tolbutamide, while parent compounds 1 and 9 in cell-free reactions and all metabolite levels were presented as peak areas without normalization.

### NAMPT Assay

NAMPT activity was measured using a coupled-enzyme assay previously described [Revollo, 2014]. Briefly, reactions consisting of 50 mM Tris pH 8, 0.4 mM PRPP, 2.5 mM ATP, 12 mM MgCl2, 0.02% BSA, 1 μM NAMPT, 77 nM NMNAT1, 10 mM semicarbazide, 30 μg/ml alcohol dehydrogenase were set up in a 384-well plate (50 μl/well). Compounds 9, 1, and FK866 were added to wells from 10 mM stocks in DMSO using a Tecan D300e digital dispenser. The reactions were started by addition of 50 μM NAM. Fluorescence due to NADH formation by alcohol dehydrogenase was measured in a BioTek Cytation 5 plate reader (Agilent, USA) set up for excitation at λ=340 nm and detection of emission at λ=460 nm. To counter screen for NMNAT1 or alcohol dehydrogenase inhibition, the assay was set up above but substituting 50 μM NMN (substrate for NMNAT1) for NAM. No inhibition of was observed under these conditions.

### Pharmacokinetics Studies

Six week old female CD1 mice (Charles River, Wilmington, MA) were dosed with 5 mg/kg 1 or 9 formulated as 5% DMSO/10% PEG400/85% D5W (5% dextrose in water, pH 7.4) in a volume of 0.2 ml/mouse. Animals were euthanized by inhalation overdose of CO2 at various points postdose. Blood was collected by cardiac puncture into K2EDTA tubes and kept on ice until they were spun at 9600×g for 10 min at 4 °C for plasma. Brains were harvested, rinsed briefly with PBS to remove surface adhering blood, weighed and then snap frozen in liquid nitrogen before storing along with plasma at −80 °C until processing. Brains were homogenized in a volume of PBS equal to 3× their weight in g. One hundred microliters of plasma or brain homogenate was mixed with 200 mL of methanol containing 0.15% formic acid and 37.5 ng/ml tolbutamide internal standard. Samples were vortexed for 15 seconds, incubated at RT for 10 min and then spun at 16,100×g. The resulting supernatant was spun a second time and then filtered through 0.2 μm PVDF filters and analyzed by LC-MS. Samples were quantitated in comparison to a standard curve prepared by spiking 1 or 9 into blank plasma or brain homogenate. The limit of quantitation was set as the lowest point on the standard curve that upon back-calculation was within 20% of the nominal value and was at least three-fold above the average peak area observed for blank plasma or brain. The LOQ for 1 was 1 ng/ml in plasma and 5 ng/ml in brain. The LOQ for 9 was 5 ng/ml in plasma and 10 ng/ml for brain homogenate. Brain concentrations were adjusted to ng/g wet tissue weight and then an estimate was made of drug in brain vasculature using the measured plasma concentration and subtracted from the measured brain concentration.³⁰ Pharmacokinetic parameters were calculated using the noncompartmental analysis tool with sparse sampling in Phoenix WinNonlin (Certara Corp, Princeton, NJ).

### IMPDH Assay

IMPDH activity was measured by monitoring NADH production through the change in absorbance at 399 nm as previously described [PMID: 13428767]. Reactions were set up in triplicate on a 384-well μClear microplate (Greiner Bio-One), each containing 0.5 ug recombinant human inosine monophosphate dehydrogenase 2 (IMPDH2, R&D Systems), 500 uM NAD, 250 uM inosine monophosphate (IMP) in 50 mM Tris pH 8.0, 300 mM NaCl, 1 mM EDTA, 1 mM DTT. Absorbance was measured every 30 seconds for 30 minutes on a BioTek Cytation 5 plate reader (Agilent, USA) at 37 °C. IMPDH2 activity was determined from the slope of absorbance vs. time plots.

The effect of 9 metabolites on IMPDH activity was tested by adding cell-free reaction mixtures prepared as described in the “Cell-free production of 1 and 9 metabolites” methods section, with few modifications. Briefly, 1 ml reactions were incubated at 37 °C overnight, quenched with one volume of methanol and vortexed for 15 seconds. After incubation for ten minutes at room temperature, samples were centrifuged at 13,200 rpm for 5 minutes. The supernatant was dried in a vacuum concentrator and resuspended in 0.5 ml of water. The reconstituted reaction and controls without compound 9 or NAMPT/NMNAT1 that were similarly prepared, were added to IMPDH reactions at 40% v/v.

### REFERENCES

1. Valentin, T., Le Cesne, A., Ray-Coquard, I., Italiano, A., Decanter, G., Bompas, E., Isambert, N., Thariat, J., Linassier, C., Bertucci, F., et al. (2016). Management and prognosis of malignant peripheral nerve sheath tumors: The experience of the French Sarcoma Group (GSF-GETO). Eur J Cancer 56, 77-84. 10.1016/j.ejca.2015.12.015.
2. Delgado-Lopez, P. D., and Corrales-Garcia, E. M. (2016). Survival in glioblastoma: a review on the impact of treatment modalities. Clin Transl Oncol 18, 1062-1071. 10.1007/s12094-016-1497-x.
3. Stupp, R., Mason, W. P., van den Bent, M. J., Weller, M., Fisher, B., Taphoorn, M. J., Belanger, K., Brandes, A. A., Marosi, C., Bogdahn, U., et al. (2005). Radiotherapy plus concomitant and adjuvant temozolomide for glioblastoma. N Engl J Med 352, 987-996. 10.1056/NEJMoa043330.
4. Chau, V., Lim, S. K., Mo, W., Liu, C., Patel, A. J., McKay, R. M., Wei, S., Posner, B. A., De Brabander, J. K., Williams, N. S., et al. (2014). Preclinical therapeutic efficacy of a novel pharmacologic inducer of apoptosis in malignant peripheral nerve sheath tumors. Cancer Res 74, 586-597. 10.1158/0008-5472.Can-13-1934.
5. Odum, E. P., and Wakwe, V. C. (2012). Plasma concentrations of water-soluble vitamins in metabolic syndrome subjects. Niger J Clin Pract 15, 442-447. 10.4103/1119-3077.104522.
6. Niehoff, I. D., Huther, L., and Lebzien, P. (2009). Niacin for dairy cattle: a review. Br J Nutr 101, 5-19. 10.1017/S0007114508043377.
7. Houtkooper, R. H., Canto, C., Wanders, R. J., and Auwerx, J. (2010). The secret life of NAD+: an old metabolite controlling new metabolic signaling pathways. Endocr Rev 31, 194-223. 10.1210/er.2009-0026.
8. Hasmann, M., and Schemainda, I. (2003). FK866, a highly specific noncompetitive inhibitor of nicotinamide phosphoribosyltransferase, represents a novel mechanism for induction of tumor cell apoptosis. Cancer Res 63, 7436-7442.
9. Khan, J. A., Tao, X., and Tong, L. (2006). Molecular basis for the inhibition of human NMPRTase, a novel target for anticancer agents. Nat Struct Mol Biol 13, 582-588. 10.1038/nsmb 1105.
10. Revollo, J. R., Grimm, A. A., and Imai, S. (2004). The NAD biosynthesis pathway mediated by nicotinamide phosphoribosyltransferase regulates Sir2 activity in mammalian cells. J Biol Chem 279, 50754-50763. 10.1074/jbc.M408388200.
11. Sanson, K. R., Hanna, R. E., Hegde, M., Donovan, K. F., Strand, C., Sullender, M. E., Vaimberg, E. W., Goodale, A., Root, D. E., Piccioni, F., and Doench, J. G. (2018). Optimized libraries for CRISPR-Cas9 genetic screens with multiple modalities. Nat Commun 9, 5416. 10.1038/s41467-018-07901-8.
12. Nguyen, T. P., Fang, M., Kim, J., Wang, B., Lin, E., Khivansara, V., Barrows, N., Rivera-Cancel, G., Goralski, M., Cervantes, C. L., et al. (2023). Inducible mismatch repair streamlines forward genetic approaches to target identification of cytotoxic small molecules. bioRxiv. 10.1101/2023.02.21.529401.
13. Buonvicino, D., Mazzola, F., Zamporlini, F., Resta, F., Ranieri, G., Camaioni, E., Muzzi, M., Zecchi, R., Pieraccini, G., Dolle, C., et al. (2018). Identification of the Nicotinamide Salvage Pathway as a New Toxification Route for Antimetabolites. Cell Chem Biol 25, 471-482 e477. 10.1016/j.chembiol.2018.01.012.
14. Loreto, A., Angeletti, C., Gu, W., Osborne, A., Nieuwenhuis, B., Gilley, J., Merlini, E., Arthur-Farraj, P., Amici, A., Luo, Z., et al. (2021). Neurotoxin-mediated potent activation of the axon degeneration regulator SARM1. Elife 10. 10.7554/eLife.72823.
15. Wu, T., Zhu, J., Strickland, A., Ko, K. W., Sasaki, Y., Dingwall, C. B., Yamada, Y., Figley, M. D., Mao, X., Neiner, A., et al. (2021). Neurotoxins subvert the allosteric activation mechanism of SARM1 to induce neuronal loss. Cell Rep 37, 109872. 10.1016/j.celrep.2021.109872.
16. Jayaram, H. N., Pillwein, K., Lui, M. S., Faderan, M. A., and Weber, G. (1986). Mechanism of resistance to tiazofurin in hepatoma 3924A. Biochem Pharmacol 35, 587-593. 10.1016/0006-2952(86)90352-7.
17. Zhou, T., Kurnasov, O., Tomchick, D. R., Binns, D. D., Grishin, N. V., Marquez, V. E., Osterman, A. L., and Zhang, H. (2002). Structure of human nicotinamide/nicotinic acid mononucleotide adenylyltransferase. Basis for the dual substrate specificity and activation of the oncolytic agent tiazofurin. J Biol Chem 277, 13148-13154. 10.1074/jbc.M111469200.
18. Grem, J. L., Rubinstein, L., King, S. A., Cheson, B. D., Hawkins, M. J., and Shoemaker, D. D. (1990). Clinical toxicity associated with tiazofurin. Invest New Drugs 8, 227-238. 10.1007/bf00177266.
19. Naffouje, R., Grover, P., Yu, H., Sendilnathan, A., Wolfe, K., Majd, N., Smith, E. P., Takeuchi, K., Senda, T., Kofuji, S., and Sasaki, A. T. (2019). Anti-Tumor Potential of IMP Dehydrogenase Inhibitors: A Century-Long Story. Cancers (Basel) 11. 10.3390/cancers11091346.
20. Tricot, G., Jayaram, H. N., Weber, G., and Hoffman, R. (1990). Tiazofurin: biological effects and clinical uses. Int J Cell Cloning 8, 161-170. 10.1002/stem.5530080303.
21. Pont, A., Rubino, J. M., Bishop, D., and Peal, R. (1979). Diabetes mellitus and neuropathy following Vacor ingestion in man. Arch Intern Med 139, 185-187.
22. Hicks, S. P. (1955). Pathologic effects of antimetabolites. I. Acute lesions in the hypothalamus, peripheral ganglia, and adrenal medulla caused by 3-acetyl pyridine and prevented by nicotinamide. Am J Pathol 31, 189-199.
23. Wang, X., Yang, K., Xie, Q., Wu, Q., Mack, S. C., Shi, Y., Kim, L. J. Y., Prager, B. C., Flavahan, W. A., Liu, X., et al. (2017). Purine synthesis promotes maintenance of brain tumor initiating cells in glioma. Nat Neurosci 20, 661-673. 10.1038/nn.4537.
24. Kofuji, S., Hirayama, A., Eberhardt, A. O., Kawaguchi, R., Sugiura, Y., Sampetrean, O., Ikeda, Y., Warren, M., Sakamoto, N., Kitahara, S., et al. (2019). IMP dehydrogenase-2 drives aberrant nucleolar activity and promotes tumorigenesis in glioblastoma. Nat Cell Biol 21, 1003-1014. 10.1038/s41556-019-0363-9.
25. Tateishi, K., Wakimoto, H., Iafrate, A. J., Tanaka, S., Loebel, F., Lelic, N., Wiederschain, D., Bedel, O., Deng, G., Zhang, B., et al. (2015). Extreme Vulnerability of IDH1 Mutant Cancers to NAD+Depletion. Cancer Cell 28, 773-784. 10.1016/j.ccell.2015.11.006.
26. Lin, J. B., Kubota, S., Ban, N., Yoshida, M., Santeford, A., Sene, A., Nakamura, R., Zapata, N., Kubota, M., Tsubota, K., et al. (2016). NAMPT-Mediated NAD(+) Biosynthesis Is Essential for Vision In Mice. Cell Rep 17, 69-85. 10.1016/j.celrep.2016.08.073.
27. Li, H., and Durbin, R. (2009). Fast and accurate short read alignment with Burrows-Wheeler transform. Bioinformatics 25, 1754-1760. 10.1093/bioinformatics/btp324.
28. Li, H., Handsaker, B., Wysoker, A., Fennell, T., Ruan, J., Homer, N., Marth, G., Abecasis, G., Durbin, R., and Genome Project Data Processing, S. (2009). The Sequence Alignment/Map format and SAMtools. Bioinformatics 25, 2078-2079. 10.1093/bioinformatics/btp352.
29. Li, W., Xu, H., Xiao, T., Cong, L., Love, M. I., Zhang, F., Irizarry, R. A., Liu, J. S., Brown, M., and Liu, X. S. (2014). MAGeCK enables robust identification of essential genes from genome-scale CRISPR/Cas9 knockout screens. Genome Biol 15, 554. 10.1186/s13059-014-0554-4.
30. Kwon, Y. (2013). Handbook of Essential Pharmacokinetics, Pharmacodynamics, and Drug Metabolism for Industrial Scientists., 1 Edition (Springer Science+Business).

### APPENDIX: SYNTHESIS, CHARACTERIZATION AND ACTIVITY OF MICROMOLAR AND NANOMOLAR ACTIVE NICOTINAMIDE DERIVATIVES

Activity: Activity of 1-66 was confirmed by IC50 (mM), wth nicotinamide at 0 uM, 33 uM 100 uM (assay details, supra).

Synthesis and characterization: Unless otherwise specified, all commercial reagents were used as received. All reactions using dried solvents were carried out under an atmosphere of argon in flame-dried glassware with magnetic stirring. Dry solvent was dispensed from a solvent purification system that passes solvent through two columns of dry neutral alumina. Silica gel chromatographic purifications were performed by flash chromatography with silica gel (Sigma, grade 60, 230-400 mesh) packed in glass columns (the eluting solvent was determined by thin layer chromatography, TLC), or with an Isco Combiflash system using RedisepRf Flash columns with size ranging from 4 to 80 grams. Analytical TLC was performed on glass plates coated with 0.25 mm silica gel (Merck Kieselgel 60F254) using UV (254 nm) for visualization. Melting points are uncorrected. Routine ¹H and proton-decoupled ¹³C NMR spectra were obtained on an Agilent or Bruker NMR spectrometer with samples at room temperature. Chemical shifts (6) are reported in parts per million (ppm) from low to high field relative to residual solvent. Multiplicities are given as: s (singlet), d (doublet), t (triplet), q (quartet), dd (doublet of doublets), m (multiplet). The purity of all tested compounds was determined by LC-MS analysis performed on an Agilent 1290 HPLC system using an Eclipse XDB-C18 column (4.6×150 mm, 5 μm; Agilent) that was coupled to an Agilent 6130 mass spectrometer run in the ESI mode in both positive and negative ionization with a scan range of 100 to 1,100 m/z. Liquid chromatography was carried out at a flow rate of 0.5 mL/min at 20 °C with a 5 μL injection volume, using the gradient elution with aqueous acetonitrile containing 0.1% formic acid. The gradient was adjusted based on the different polarity of different compounds.

General Procedure A. A mixture of the corresponding carboxylic acid (1 equivalent), 4-dimethylaminopyridine (DMAP, 0.25 equivalents), and 1-ethyl-3-(3-dimethylaminopropyl)carbodiimide hydrochloride (EDC, 1.12 equivalents) in CH₂Cl₂ (~5 mL/mmol) was stirred at rt for 5-15 min followed by the addition of the corresponding amine (1.1 equivalents). The resulting solution was stirred for 12 to 30 h (progress monitored by TLC) at rt and was diluted with CH₂Cl₂ (~5-10 times reaction volume), washed with brine (equal volume) and aq. sat. NaHCO₃ (equal volume). The organic layer was dried over Na₂SO₄, filtered and concentrated under reduced pressure. The residue was purified via flash chromatography on silica gel (methanol/CH₂Cl₂ or EtOAc/hexanes) to afford the product.

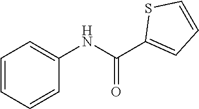 [SMILES: O=C(Nc1ccccc1)c1cccs1]

N-Phenylthiophene-2-carboxamide (2). Obtained as a white solid according to general method A (83% yield). ¹H NMR (400 MHz, CDCl₃) δ 7.88 (s, 1H), 7.64 (d, J=3.8 Hz, 1H), 7.61 (d, J=7.8 Hz, 2H), 7.53 (d, J=5.1 Hz, 1H), 7.34 (t, J=7.9 Hz, 2H), 7.18-7.06 (m, 2H). ¹³C NMR (100 MHz, CDCl₃) δ 160.0, 139.3, 137.6, 130.8, 129.1, 128.5, 127.8, 124.6, 120.3. [MH]⁺ 199.2. Purity (LC-MS): 98.3%.

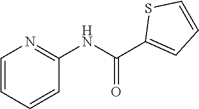 [SMILES: O=C(Nc1ccccn1)c1cccs1]

N-(Pyridin-2-yl)thiophene-2-carboxamide (3). Obtained as a white solid according to general method A (78% yield). ¹H NMR (400 MHz, CDCl₃) δ 8.67 (d, J=35.8 Hz, 1H), 8.32 (d, J=8.4 Hz, 1H), 8.30-8.20 (m, 1H), 7.75 (t, J=7.4 Hz, 1H), 7.68 (d, J=3.8 Hz, 1H), 7.58 (d, J=5.0 Hz, 1H), 7.13 (d, J=3.6 Hz, 1H), 7.11-7.02 (m, 1H). ¹³C NMR (100 MHz, CDCl₃) δ 160.0, 151.3, 147.8, 138.6, 131.6, 128.9, 128.0, 120.0, 114.3, 114.2. [MH]⁺ 205.1. Purity (LC-MS): 95%.

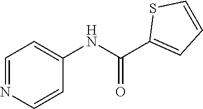 [SMILES: O=C(Nc1ccncc1)c1cccs1]

N-(Pyridin-4-yl)thiophene-2-carboxamide (4). The title product was obtained via the reaction conditions in general method A. Instead of aqueous work up, the reaction mixture was concentrated under reduced pressure, followed by the addition of Et₂O and sonification. After settling, the ether was removed via pipette, and the precipitate was purified by flash chromatography on silica gel (MeOH/CH₂Cl₂ 1:19) to provide the product as a white solid (11% yield). ¹H NMR (400 MHz, CD₃OD) δ 8.41 (d, J=6.7 Hz, 2H), 7.95 (dd, J=3.9, 1.1 Hz, 1H), 7.83-7.79 (m, 2H), 7.77 (dd, J=5.0, 1.1 Hz, 1H), 7.19 (dd, J=5.0, 3.8 Hz, 1H). ¹³C NMR (100 MHz, CD₃OD) δ 161.6, 149.3, 146.8, 138.7, 132.2, 129.5, 127.7, 114.2. [MH]⁺ 205.1. Purity (LC-MS): 98.2%.

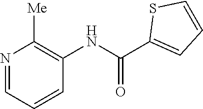 [SMILES: Cc1ncccc1NC(=O)c1cccs1]

N-(2-Methylpyridin-3-yl)thiophene-2-carboxamide (5). Obtained as a light yellow gel according to general method A (65% yield). ¹H NMR (400 MHz, CDCl₃) δ 8.35-8.27 (m, 1H), 8.29-8.19 (m, 1H), 7.77-7.61 (m, 2H), 7.61-7.54 (m, 1H), 7.22-7.17 (m, 1H), 7.17-7.10 (m, 1H), 2.72-2.31 (m, 3H). ¹³C NMR (100 MHz, CDCl₃) δ 160.1, 149.4, 145.5, 138.5, 132.1, 131.2, 130.1, 128.9, 128.0, 121.9, 20.7. [MH]⁺ 219.1. Purity (LC-MS): 94.7%.

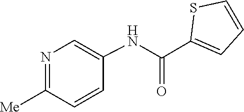 [SMILES: Cc1ccc(NC(=O)c2cccs2)cn1]

N-(6-Methylpyridin-3-yl)thiophene-2-carboxamide (6). Obtained as a white solid according to general method A (81% yield). ¹H NMR (400 MHz, CDCl₃) δ 8.51 (d, J=2.6 Hz, 1H), 8.18 (s, 1H), 8.10 (dd, J=8.4, 2.7 Hz, 1H), 7.68 (d, J=3.8 Hz, 1H), 7.55 (d, J=5.0 Hz, 1H), 7.14 (d, J=8.4 Hz, 1H), 7.10 (dd, J=5.0, 3.8 Hz, 1H), 2.51 (s, 3H). ¹³C NMR (100 MHz, CDCl3) δ 160.4, 154.3, 140.8, 138.6, 132.1, 131.2, 128.9, 128.5, 127.9, 123.3, 23.8. [MH]⁺ 219.1. Purity (LC-MS): 94.7%

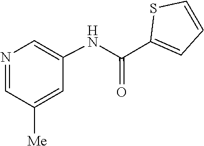 [SMILES: Cc1cncc(NC(=O)c2cccs2)c1]

N-(5-Methylpyridin-3-yl)thiophene-2-carboxamide (7). Obtained as a white solid according to general method A (75% yield). ¹H NMR (400 MHz, CDCl₃) δ 8.54 (s, 1H), 8.42 (s, 1H), 8.17 (s, 1H), 8.09 (s, 1H), 7.70 (d, J=3.7 Hz, 1H), 7.55 (d, J=4.9 Hz, 1H), 7.09 (t, J=4.2 Hz, 1H), 2.31 (s, 3H). ¹³C NMR (100 MHz, CDCl₃) δ 160.6, 145.9, 138.7, 134.4, 133.8, 131.4, 129.0, 128.5, 127.9, 18.4. [MH]⁺ 219.1. Purity (LC-MS): 94.7%.

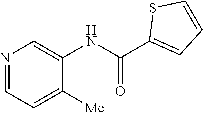 [SMILES: Cc1ccncc1NC(=O)c1cccs1]

N-(4-Methylpyridin-3-yl)thiophene-2-carboxamide (8). Obtained as a light yellow gel according to general method A (77% yield). ¹H NMR (400 MHz, CDCl₃) δ 8.83 (s, 1H), 8.34 (d, J=4.5 Hz, 1H), 7.77 (s, 1H), 7.68 (d, J=2.7 Hz, 1H), 7.58 (d, J=4.4 Hz, 1H), 7.22-7.10 (m, 2H), 2.33 (s, 3H). ¹³C NMR (100 MHz, CDCl3) δ 160.2, 146.9, 145.8, 140.6, 138.1, 132.5, 131.1, 129.2, 128.0, 125.4, 17.6. [MH]⁺ 219.1. Purity (LC-MS): 95%

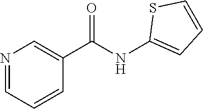 [SMILES: O=C(Nc1cccs1)c1cccnc1]

N-(Thiophen-2-yl)nicotinamide (9). A mixture of nicotinic acid (0.139 g, 1.13 mmol) and SOCl2 (2 mL) was stirred at 40 °C for 17 h and then at 60 °C for 2 h. After evaporation of excess SOCl2, the nicotinoyl chloride HCl salt was obtained and used for next step without further purification.

A mixture of tert-butyl thiophen-2-ylcarbamate (0.200 g, 1.00 mmol), CH₂Cl₂ (6 mL) and CF₃CO₂H (TFA, 1.5 mL, 2.24 g, 19.6 mmol) was stirred at rt for 2 h. After evaporation of excess CF₃CO₂H and CH₂Cl₂, the thiophen-2-amine TFA salt was obtained and used as is.

To a flask were added the nicotinoyl chloride HCl salt, thiophen-2-amine TFA salt, CH₂Cl₂ (3.5 mL), water (3.5 mL) and K2CO3 (1.1552 g, 8.37 mmol) at 0 °C. The resulting biphasic solution was warmed to rt and stirred vigorously for 20 h. The reaction mixture was diluted with brine (60 mL) and extracted with CH₂Cl₂ (2×60 mL). The combined organic layers were dried over Na2SO4, filtered and concentrated under reduced pressure. The residue was purified via flash chromatography on silica gel (1:19 methanol/CH₂Cl₂) to afford the title product (0.043 g, 21%) as a light brown solid. ¹H NMR (400 MHz, CD₃OD) δ 9.10 (d, J=1.4 Hz, 1H), 8.72 (d, J=5.0 Hz, 1H), 8.37 (d, J=8.1 Hz, 1H), 7.59 (dd, J=7.7, 5.3 Hz, 1H), 6.99 (dd, J=5.4, 1.5 Hz, 1H), 6.96-6.86 (m, 2H). ¹³C NMR (100 MHz, CD₃OD) δ 162.5, 151.6, 147.9, 139.2, 135.9, 129.8, 123.8, 123.7, 117.8, 112.8. [MH]⁺ 205.1. Purity (LC-MS): 95.6%

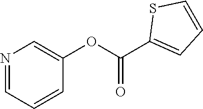 [SMILES: O=C(Oc1cccnc1)c1cccs1]

Pyridin-3-yl thiophene-2-carboxylate (10). Obtained as a white solid according to general method A (95% yield). ¹H NMR (400 MHz, CDCl₃) δ 8.55 (s, 1H), 8.51 (dd, J=4.7, 1.6 Hz, 1H), 8.00 (s, 1H), 7.70 (dt, J=4.9, 1.6 Hz, 1H), 7.60 (ddd, J=8.3, 2.8, 1.5 Hz, 1H), 7.37 (ddd, J=8.1, 4.7, 2.5 Hz, 1H), 7.19 (dt, J=5.1, 3.1 Hz, 1H). ¹³C NMR (100 MHz, CDCl₃) δ 160.0, 147.3, 147.1, 143.5, 135.2, 134.2, 131.9, 129.3, 128.2, 123.9. [MH]⁺ 206.1. Purity (LC-MS): 97.4%.

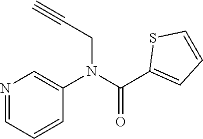 [SMILES: C#CCN(C(=O)c1cccs1)c1cccnc1]

N-(Prop-2-yn-1-yl)-N-(pyridin-3-yl)thiophene-2-carboxamide (11). N-(pyridin-3-yl)thiophene-2-carboxamide (1, 0.156 g, 0.76 mmol) was dissolved in anhydrous DMF (2.5 mL), followed by the addition of NaH (60%, 0.063 g, 1.57 mmol) at 0 °C. The flask was immediately blanketed with argon and sealed by a rubber septum fitted with an argon balloon. The reaction solution was stirred for 7 min at 0 °C and propargyl bromide (80% in toluene, 0.10 mL, 0.90 mmol) was added via syringe. The resulting solution was stirred for 2 h at 0 °C and the reaction was quenched by slowly adding brine (10 mL) and then EtOAc (100 mL). The resulting bi-phasic solution was washed by 25% LiCl solution (3×100 mL) and brine (100 mL). The organic layer was dried over Na2SO4, filtered and concentrated under reduced pressure. Purification of the residue via flash chromatography on silica gel (1:9 CH₃OH/CH₂Cl₂) provided the desired title compound as a brown gel (0.060 g, 32%). ¹H NMR (400 MHz, CDCl₃) δ 8.66 (d, J=4.8 Hz, 1H), 8.59 (s, 1H), 7.69 (dd, J=8.1, 1.4 Hz, 1H), 7.40 (dd, J=8.2, 4.9 Hz, 1H), 7.35 (d, J=5.1 Hz, 1H), 6.96-6.90 (m, 1H), 6.87-6.79 (m, 1H), 4.65 (t, J=2.0 Hz, 2H), 2.29 (t, J=2.5 Hz, 1H). ¹³C NMR (100 MHz, CDCl₃) δ 162.2, 150.1, 149.6, 138.5, 136.4, 133.3, 131.6, 126.9, 124.2, 78.1, 73.4, 40.2. [MH]⁺ 243.1. Purity (LC-MS): 95.4%.

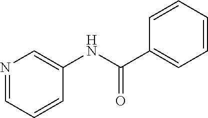 [SMILES: O=C(Nc1cccnc1)c1ccccc1]

N-(Pyridin-3-yl)benzamide (12). Obtained as a white solid according to general method A (80% yield). ¹H NMR (400 MHz, CDCl₃) δ 8.67 (d, J=11.6 Hz, 2H), 8.33-8.22 (m, 2H), 7.87 (d, J=8.6 Hz, 2H), 7.53 (t, J=7.4 Hz, 1H), 7.44 (t, J=7.5 Hz, 2H), 7.27 (dd, J=8.4, 5.0 Hz, 1H). ¹³C NMR (100 MHz, CDCl₃) δ 166.5, 145.3, 141.6, 135.1, 134.2, 132.2, 128.8, 127.9, 127.2, 123.8. [MH]⁺ 199.2. Purity (LC-MS): 98.3%.

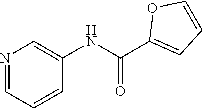 [SMILES: O=C(Nc1cccnc1)c1ccco1]

N-(Pyridin-3-yl)furan-2-carboxamide (13). Obtained as a white solid according to general method A (63% yield). ¹H NMR (400 MHz, CDCl₃) δ 8.71 (d, J=2.6 Hz, 1H), 8.38 (d, J=4.3 Hz, 1H), 8.34-8.16 (m, 2H), 7.53 (s, 1H), 7.31 (dd, J=8.4, 4.7 Hz, 1H), 7.27 (d, J=3.7 Hz, 1H), 6.62-6.52 (m, 1H). ¹³C NMR (100 MHz, CDCl₃) δ 156.4, 147.2, 145.5, 144.6, 141.3, 134.3, 127.2, 123.8, 115.9, 112.8. [MH]⁺ 189.1. Purity (LC-MS): 99.5%.

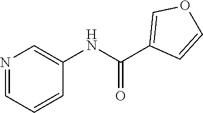 [SMILES: O=C(Nc1cccnc1)c1ccoc1]

N-(Pyridin-3-yl)furan-3-carboxamide (14). Obtained as a white solid according to general method A (32% yield). ¹H NMR (400 MHz, CDCl₃) δ 8.63 (d, J=2.6 Hz, 1H), 8.35 (d, J=4.7 Hz, 1H), 8.26 (d, J=8.6 Hz, 1H), 8.09 (s, 2H), 7.48 (s, 1H), 7.30 (dd, J=8.4, 4.8 Hz, 1H), 6.78 (s, 1H). ¹³C NMR (100 MHz, CDCl₃) δ 161.2, 145.6, 145.3, 144.2, 141.3, 134.7, 127.9, 123.9, 122.5, 108.3 [MH]⁺ 189.1. Purity (LC-MS): 95.5%.

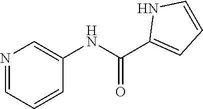 [SMILES: O=C(Nc1cccnc1)c1ccc[nH]1]

N-(Pyridin-3-yl)-1H-pyrrole-2-carboxamide (15). The title product was obtained via the reaction conditions in general method A. Instead of aqueous work up, the reaction mixture was concentrated under reduced pressure, followed by the addition of CH₂Cl₂Et₂O) (1:1) and sonification. After settling, the precipitate was filtered to provide the product as a yellow solid (43% yield). ¹H NMR (400 MHz, DMSO-d6) δ 11.74 (s, 1H), 9.94 (s, 1H), 8.88 (s, 1H), 8.24 (d, J=4.8 Hz, 1H), 8.13 (d, J=8.5 Hz, 1H), 7.34 (dd, J=8.3, 4.7 Hz, 1H), 7.08 (s, 1H), 6.98 (s, 1H), 6.18 (s, 1H). ¹³C NMR (100 MHz, DMSO-d6) δ 159.9, 144.3, 141.9, 136.5, 127.2, 126.0, 123.9, 123.5, 112.2, 109.5. [MH]⁺ 188.1. Purity (LC-MS): 98%.

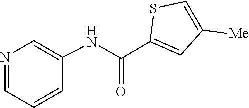 [SMILES: Cc1csc(C(=O)Nc2cccnc2)c1]

4-Methyl-N-(pyridin-3-yl)thiophene-2-carboxamide (16). Obtained as a light yellow gel according to general method A (85% yield). ¹H NMR (400 MHz, CDCl₃) δ 8.64 (d, J=2.7 Hz, 1H), 8.35 (d, J=4.7 Hz, 1H), 8.24 (d, J=8.4 Hz, 1H), 8.21 (s, 1H), 7.50 (s, 1H), 7.28 (dd, J=8.4, 4.8 Hz, 1H), 7.14 (s, 1H), 2.27 (s, 3H). ¹³C NMR (100 MHz, CDCl3) δ 160.6, 145.4, 141.4, 138.7, 137.8, 134.7, 131.2, 127.7, 127.0, 123.8, 15.7. [MH]⁺ 219.1. Purity (LC-MS): 97.4%.

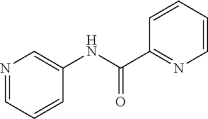 [SMILES: O=C(Nc1cccnc1)c1ccccn1]

N-(Pyridin-3-yl)picolinamide (17). Obtained as a white solid according to general method A (67% yield). ¹H NMR (400 MHz, CDCl₃) δ 10.10 (s, 1H), 8.83 (d, J=2.5 Hz, 1H), 8.63 (d, J=4.1 Hz, 1H), 8.44-8.36 (m, 2H), 8.29 (d, J=7.9 Hz, 1H), 7.93 (td, J=7.7, 1.7 Hz, 1H), 7.51 (ddd, J=7.6, 4.8, 1.2 Hz, 1H), 7.33 (dd, J=8.2, 4.9 Hz, 1H). ¹³C NMR (100 MHz, CDCl3) δ 162.5, 149.1, 148.1, 145.4, 141.3, 137.8, 134.5, 126.8, 126.6, 123.7, 122.5. [MH]⁺ 200.1. Purity (LC-MS): 97.6%.

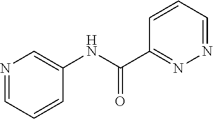 [SMILES: O=C(Nc1cccnc1)c1cccnn1]

N-(Pyridin-3-yl)pyridazine-3-carboxamide (18). Obtained as a white solid according to general method A (27% yield). ¹H NMR (400 MHz, CDCl₃) δ 11.35 (s, 1H), 9.47 (dd, J=5.0, 1.7 Hz, 1H), 9.09 (d, J=2.5 Hz, 1H), 8.56-8.23 (m, 3H), 7.98 (dd, J=8.5, 5.0 Hz, 1H), 7.41 (dd, J=8.3, 4.6 Hz, 1H). ¹³C NMR (100 MHz, CDCl3) δ 162.5, 154.1, 153.3, 145.6, 142.9, 135.4, 129.2, 128.2, 126.7, 124.0. [MH]⁺ 201.1. Purity (LC-MS): 97.6%.

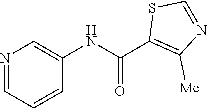 [SMILES: Cc1ncsc1C(=O)Nc1cccnc1]

4-Methyl-N-(pyridin-3-yl)thiazole-5-carboxamide (19). Obtained as a light yellow gel according to general method A (59% yield). ¹H NMR (400 MHz, CDCl₃) δ 8.77 (s, 1H), 8.62 (s, 1H), 8.38 (s, 1H), 8.20 (d, J=8.4 Hz, 1H), 7.98 (s, 1H), 7.32 (dd, J=8.4, 4.6 Hz, 1H), 2.79 (s, 3H). ¹³C NMR (100 MHz, CDCl3) δ 160.3, 157.4, 153.0, 145.9, 141.6, 134.4, 128.0, 125.3, 123.8, 17.4. [MH]⁺ 220.1. Purity (LC-MS): 89%.

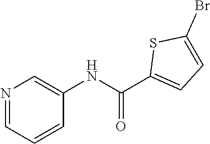 [SMILES: O=C(Nc1cccnc1)c1ccc(Br)s1]

5-Bromo-N-(pyridin-3-yl)thiophene-2-carboxamide (20). The title product was obtained via the reaction conditions in general method A. Instead of aqueous work up, the reaction mixture was concentrated under reduced pressure, followed by the addition of MeOH/Et₂O (1:1) and sonification. The resulting suspension was filtered, and the precipitate (cake) was collected as a white solid (24% yield). ¹H NMR (400 MHz, DMSO-d6) δ 10.51 (s, 1H), 8.85 (d, J=2.6 Hz, 1H), 8.31 (d, J=4.7 Hz, 1H), 8.09 (d, J=8.8 Hz, 1H), 7.88 (d, J=4.1 Hz, 1H), 7.38 (q, J=4.5, 3.7 Hz, 2H). ¹³C NMR (100 MHz, DMSO-d6) δ 159.6, 145.3, 142.3, 141.5, 135.6, 132.3, 130.9, 127.9, 124.1, 118.8. Purity (LC-MS): 95.7%.

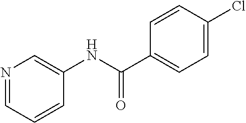 [SMILES: O=C(Nc1cccnc1)c1ccc(Cl)cc1]

4-Chloro-N-(pyridin-3-yl)benzamide (21). Obtained as a white solid according to general method A (81% yield). ¹H NMR (400 MHz, CD₃OD) δ 8.87 (d, J=2.5 Hz, 1H), 8.30 (dd, J=4.8, 1.4 Hz, 1H), 8.24 (ddd, J=8.4, 2.5, 1.4 Hz, 1H), 7.94 (d, J=8.6 Hz, 2H), 7.53 (d, J=8.6 Hz, 2H), 7.44 (dd, J=8.4, 4.8 Hz, 1H). ¹³C NMR (100 MHz, CD₃OD) δ 166.3, 144.1, 141.3, 137.9, 136.1, 132.8, 129.0, 128.6, 128.5, 123.9. [MH]⁺ 233.1. Purity (LC-MS): 95%.

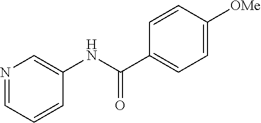 [SMILES: COc1ccc(C(=O)Nc2cccnc2)cc1]

4-Methoxy-N-(pyridin-3-yl)benzamide (22). Obtained as a white solid according to general method A (79% yield). ¹H NMR (400 MHz, CD₃OD) δ 8.87 (d, J=2.6 Hz, 1H), 8.28 (dd, J=4.9, 1.4 Hz, 1H), 8.23 (ddd, J=8.4, 2.5, 1.4 Hz, 1H), 7.94 (d, J=8.8 Hz, 2H), 7.42 (dd, J=8.4, 4.8 Hz, 1H), 7.03 (d, J=8.9 Hz, 2H), 3.86 (s, 3H). ¹³C NMR (100 MHz, CD₃OD) δ 167.1, 163.0, 143.8, 141.4, 136.3, 129.3, 128.6, 126.0, 123.8, 113.5, 54.6. [MH]⁺ 229.1. Purity (LC-MS): 89%.

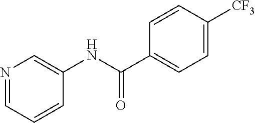 [SMILES: O=C(Nc1cccnc1)c1ccc(C(F)(F)F)cc1]

N-(Pyridin-3-yl)-4-(trifluoromethyl)benzamide (23). Obtained as a light yellow solid according to general method A (86% yield). ¹H NMR (400 MHz, CD₃OD) δ 8.90 (d, J=2.6 Hz, 1H), 8.32 (dd, J=4.9, 1.4 Hz, 1H), 8.27 (ddd, J=8.4, 2.6, 1.5 Hz, 1H), 8.13 (d, J=7.9 Hz, 2H), 7.84 (d, J=8.3 Hz, 2H), 7.46 (dd, J=8.4, 4.9 Hz, 1H). ¹³C NMR (100 MHz, CD₃OD) δ 166.1, 144.3, 141.4, 138.0, 136.0, 133.1 (q, J=32.4 Hz), 128.6, 128.1, 125.2 (q, J=3.8 Hz), 123.9, 122.5. [MH]⁺ 267.1. Purity (LC MS): 97.1%.

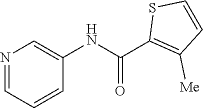 [SMILES: Cc1ccsc1C(=O)Nc1cccnc1]

3-Methyl-N-(pyridin-3-yl)thiophene-2-carboxamide (24). Obtained as a light yellow gel according to general method A (40% yield). ¹H NMR (400 MHz, CDCl₃) δ 8.61 (d, J=2.7 Hz, 1H), 8.37 (d, J=5.1 Hz, 1H), 8.22 (d, J=8.4 Hz, 1H), 7.71 (s, 1H), 7.36 (d, J=5.0 Hz, 1H), 7.31 (dd, J=8.4, 4.7 Hz, 1H), 6.96 (d, J=5.0 Hz, 1H), 2.59 (s, 3H). ¹³C NMR (100 MHz, CDCl₃) δ 161.5, 145.5, 143.3, 141.5, 134.6, 132.5, 129.8, 127.6, 127.4, 123.7, 16.0. [MH]⁺ 219.1. Purity (LC-MS): 89%.

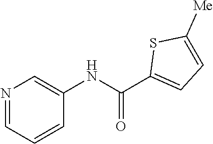 [SMILES: Cc1ccc(C(=O)Nc2cccnc2)s1]

5-Methyl-N-(pyridin-3-yl)thiophene-2-carboxamide (25). Obtained as a white solid according to general method A (54% yield). ¹H NMR (400 MHz, CD₃OD) δ 8.83 (d, J=2.6 Hz, 1H), 8.27 (dd, J=4.8, 1.5 Hz, 1H), 8.19 (ddd, J=8.4, 2.6, 1.5 Hz, 1H), 7.72 (d, J=3.8 Hz, 1H), 7.41 (dd, J=8.4, 4.8 Hz, 1H), 6.86 (dd, J=3.8, 1.1 Hz, 1H), 2.53 (s, 3H). ¹³C NMR (100 MHz, CD₃OD) δ 161.5, 147.3, 143.8, 141.1, 136.0, 129.7, 128.4, 126.3, 123.8, 14.1. Purity (LC-MS): 96.4%.

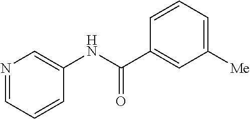 [SMILES: Cc1cccc(C(=O)Nc2cccnc2)c1]

3-Methyl-N-(pyridin-3-yl)benzamide (26). Obtained as a yellow gel according to general method A (80% yield). ¹H NMR (400 MHz, CDCl₃) δ 8.66 (d, J=2.6 Hz, 1H), 8.52-8.20 (m, 3H), 7.69 (s, 1H), 7.68-7.63 (m, 1H), 7.38-7.32 (m, 2H), 7.32-7.26 (m, 1H), 2.39 (d, J=2.7 Hz, 3H). ¹³C NMR (100 MHz, CDCl₃) δ 166.6, 145.3, 145.3, 141.5, 138.8, 138.7, 135.1, 135.0, 134.2, 133.0, 128.7, 127.9, 127.7, 127.7, 124.2, 123.8, 21.4. [MH]⁺ 213.2. Purity (LC-MS): 97.4%.

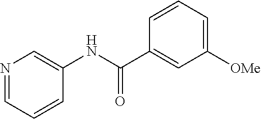 [SMILES: COc1cccc(C(=O)Nc2cccnc2)c1]

3-Methoxy-N-(pyridin-3-yl)benzamide (27). Obtained as a yellow solid according to general method A (77% yield). ¹H NMR (400 MHz, CD₃OD) δ 8.88 (d, J=1.8 Hz, 1H), 8.30 (dd, J=4.8, 1.5 Hz, 1H), 8.25 (ddd, J=8.3, 2.5, 1.4 Hz, 1H), 7.56-7.48 (m, 2H), 7.47-7.38 (m, 2H), 7.15 (ddd, J=8.2, 2.6, 1.0 Hz, 1H), 3.87 (s, 3H). ¹³C NMR (100 MHz, CD₃OD) δ 167.4, 159.9, 144.0, 141.4, 136.2, 135.5, 129.4, 128.6, 123.8, 119.4, 117.6, 112.5, 54.5. [MH]⁺ 229.1. Purity (LC-MS): 96.9%.

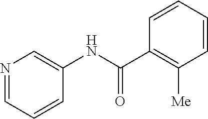 [SMILES: Cc1ccccc1C(=O)Nc1cccnc1]

2-Methyl-N-(pyridin-3-yl)benzamide (28). Obtained as a yellow solid according to general method A (69% yield). ¹H NMR (400 MHz, CDCl₃) δ 8.57 (s, 1H), 8.31 (d, J=7.9 Hz, 1H), 8.27 (d, J=4.7 Hz, 1H), 8.06 (s, 1H), 7.47 (d, J=7.5 Hz, 1H), 7.37 (t, J=7.6 Hz, 1H), 7.33-7.18 (m, 3H), 2.49 (s, 3H). ¹³C NMR (100 MHz, CDCl₃) δ 168.6, 145.4, 141.1, 136.6, 135.7, 135.0, 131.4, 130.6, 127.3, 126.7, 126.0, 123.8, 19.8. [MH]⁺ 213.1. Purity (LC-MS): 97%.

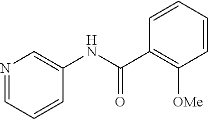 [SMILES: COc1ccccc1C(=O)Nc1cccnc1]

2-Methoxy-N-(pyridin-3-yl)benzamide (29). Obtained as a white solid according to general method A (75% yield). ¹H NMR (400 MHz, CDCl₃) δ 9.90 (s, 1H), 8.63 (d, J=2.0 Hz, 1H), 8.45-8.32 (m, 2H), 8.27 (dd, J=7.9, 1.8 Hz, 1H), 7.57-7.47 (m, 1H), 7.30 (dd, J=8.2, 4.8 Hz, 1H), 7.14 (t, J=7.6 Hz, 1H), 7.05 (d, J=8.4 Hz, 1H), 4.08 (s, 3H). ¹³C NMR (100 MHz, CDCl₃) δ 163.7, 157.2, 145.1, 141.7, 135.2, 133.7, 132.5, 127.6, 123.7, 121.8, 121.0, 111.6, 56.3. [MH]⁺ 229.1. Purity (LC-MS): 96.9%.

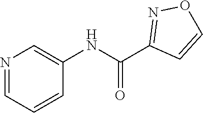 [SMILES: O=C(Nc1cccnc1)c1ccon1]

N-(Pyridin-3-yl)isoxazole-3-carboxamide (30). Obtained as a white solid according to general method A (3% yield after recrystallization from CH₂Cl₂/hexanes). ¹H NMR (400 MHz, CDCl₃) δ 8.99-8.62 (m, 2H), 8.55 (s, 1H), 8.53-8.30 (br, 1H), 8.24 (d, J=8.6 Hz, 1H), 7.43-7.28 (m, 1H), 6.92 (s, 1H). ¹³C NMR (100 MHz, CDCl₃) δ 160.5, 157.6, 156.9, 146.0, 141.4, 127.3, 123.9, 104.6. [MH]⁺ 190.1. Purity (LC-MS): 92.7%.

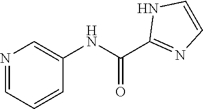 [SMILES: O=C(Nc1cccnc1)c1ncc[nH]1]

N-(Pyridin-3-yl)-1H-imidazole-2-carboxamide (31). The title product was obtained via the reaction conditions in general method A. Instead of aqueous work up, the reaction mixture was concentrated under reduced pressure, followed by the addition of Et₂O and sonification. The supernatant was removed via pipette, and the residual precipitate was resuspended in Et₂O and sonicated. Removal of the ether via pipette and drying of the remaining solid under high vacuum provided the title compound as a light yellow solid (11% yield). ¹H NMR (400 MHz, CD₃OD) δ 8.94 (d, J=2.5 Hz, 1H), 8.29 (d, J=4.8 Hz, 1H), 8.24 (d, J=8.5 Hz, 1H), 7.44 (dd, J=8.4, 4.8 Hz, 1H), 7.25 (s, 2H). ¹³C NMR (100 MHz, 4/1 CD₃OD/CDCl₃) δ 160.9, 148.0, 144.6, 144.5, 139.4, 133.2, 131.6, 128.0, 124.1. [MH]⁺ 189.1. Purity (LC-MS): 67.3%

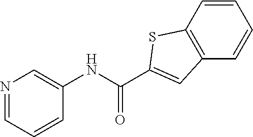 [SMILES: O=C(Nc1cccnc1)c1cc2ccccc2s1]

N-(Pyridin-3-yl)benzo[b]thiophene-2-carboxamide (32). Obtained as a light yellow solid according to general method A (87% yield). ¹H NMR (400 MHz, CD₃OD) δ 8.89 (d, J=2.6 Hz, 1H), 8.30 (dd, J=4.8, 1.5 Hz, 1H), 8.25 (ddd, J=8.3, 2.5, 1.4 Hz, 1H), 8.16 (s, 1H), 7.95-7.89 (m, 2H), 7.49-7.40 (m, 3H). ¹³C NMR (100 MHz, CD₃OD) δ 161.9, 144.1, 141.4, 141.2, 139.3, 138.6, 135.9, 128.4, 126.5, 126.1, 125.1, 124.7, 123.9, 122.2. [MH]⁺ 255.1. Purity (LC-MS): 97.3%.

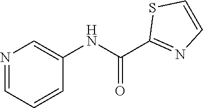 [SMILES: O=C(Nc1cccnc1)c1nccs1]

N-(Pyridin-3-yl)thiazole-2-carboxamide (33). Obtained as a light yellow solid according to general method A (4% yield after recrystallization from hexane/CH₂Cl₂). ¹H NMR (400 MHz, CDCl₃) δ 9.21 (s, 1H), 8.82 (s, 1H), 8.42 (s, 1H), 8.33 (d, J=8.5 Hz, 1H), 7.95 (d, J=3.0 Hz, 1H), 7.68 (d, J=3.1 Hz, 1H), 7.36 (dd, J=8.4, 4.7 Hz, 1H). ¹³C NMR (100 MHz, CDCl₃) δ 163.0, 157.6, 145.5, 143.7, 141.0, 134.1, 127.1, 125.8, 123.9. [MH]⁺ 206.1. Purity (LC-MS): 91.7%.

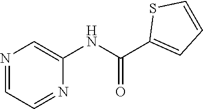 [SMILES: O=C(Nc1cnccn1)c1cccs1]

N-(Pyrazin-2-yl)thiophene-2-carboxamide (34). Obtained as a white solid according to general method A (13% yield after recrystallization from hexane/CH₂Cl₂). ¹H NMR (400 MHz, CDCl₃) δ 9.65 (s, 1H), 8.46 (s, 1H), 8.38 (d, J=2.6 Hz, 1H), 8.26 (s, 1H), 7.71 (d, J=3.8 Hz, 1H), 7.63 (d, J=5.0 Hz, 1H), 7.20-7.11 (m, 1H). ¹³C NMR (100 MHz, CDCl₃) δ 159.7, 148.0, 142.1, 140.4, 137.8, 137.2, 132.3, 129.6, 128.2. [MH]⁺ 206.1. Purity (LC-MS): 95.7%.

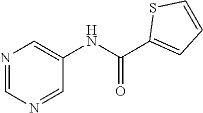 [SMILES: O=C(Nc1cncnc1)c1cccs1]

N-(Pyrimidin-5-yl)thiophene-2-carboxamide (35). Obtained as a light yellow solid according to general method A (35% yield). ¹H NMR (400 MHz, CD₃OD) δ 9.16 (s, 2H), 8.89 (s, 1H), 7.94 (dd, J=3.8, 1.2 Hz, 1H), 7.79 (dd, J=5.2, 1.1 Hz, 1H), 7.21 (dd, J=5.0, 3.8 Hz, 1H). ¹³C NMR (100 MHz, CD₃OD) δ 161.5, 152.8, 148.1, 138.1, 134.7, 132.2, 129.6, 127.7. [MH]+206.1. Purity (LC-MS): 96.4%.

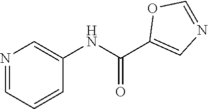 [SMILES: O=C(Nc1cccnc1)c1cnco1]

N-(Pyridin-3-yl)oxazole-4-carboxamide (36). Obtained as a white solid according to general method A (55% yield). ¹H NMR (400 MHz, CD₃OD) δ 8.92 (dd, J=2.5, 0.8 Hz, 1H), 8.57 (d, J=1.0 Hz, 1H), 8.35-8.28 (m, 2H), 8.25 (ddd, J=8.4, 2.6, 1.5 Hz, 1H), 7.44 (ddd, J=8.4, 4.8, 0.8 Hz, 1H). ¹³C NMR (100 MHz, CD₃OD) δ 159.8, 152.0, 144.3, 142.8, 141.3, 135.4, 135.3, 128.4, 123.9. [MH]⁺ 190.1. Purity (LC-MS): 74%.

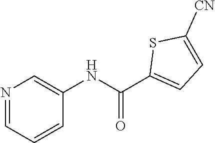 [SMILES: N#Cc1ccc(C(=O)Nc2cccnc2)s1]

5-Cyano-N-(pyridin-3-yl)thiophene-2-carboxamide (37). The title product was obtained via the reaction conditions in general method A. Instead of aqueous work up, the reaction mixture was concentrated under reduced pressure, followed by purification to afford the title product as a white solid (63%). ¹H NMR (400 MHz, 2/1 CDCl₃/CD₃OD) δ 8.55 (d, J=2.6 Hz, 1H), 8.06 (dd, J=4.8, 1.5 Hz, 1H), 8.00 (ddd, J=8.4, 2.6, 1.4 Hz, 1H), 7.66 (d, J=4.0 Hz, 1H), 7.45 (d, J=4.1 Hz, 1H), 7.15 (dd, J=8.4, 4.8 Hz, 1H). ¹³C NMR (100 MHz, 2/1 CDCl₃/CD₃OD) δ 163.46, 150.09, 148.69, 145.36, 141.93, 139.25, 132.68, 132.39, 127.99, 117.91, 117.23. [MH]⁺ 230.1. Purit (LC-MS): 94.8%.

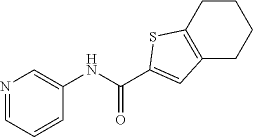 [SMILES: O=C(Nc1cccnc1)c1cc2c(s1)CCCC2]

N-(Pyridin-3-yl)-4,5,6,7-tetrahydrobenzo[b]thiophene-2-carboxamide (38). The title product was obtained via the reaction conditions in general method A. Instead of aqueous work up, the reaction mixture was concentrated under reduced pressure, followed by purification to afford the title product as a white solid (95%). ¹H NMR (400 MHz, CDCl₃) δ 8.65 (s, 1H), 8.36 (s, 1H), 8.26 (d, J=9.3 Hz, 1H), 8.11 (s, 1H), 7.38 (s, 1H), 7.30 (d, J=6.7 Hz, 1H), 2.81 (t, J=6.0 Hz, 2H), 2.61 (t, J=6.1 Hz, 2H), 1.93-1.76 (m, 4H). ¹³C NMR (100 MHz, CDCl₃) δ 160.8, 145.2, 143.1, 141.4, 136.7, 135.0, 133.8, 130.2, 127.5, 123.7, 25.4, 25.4, 23.2, 22.5. [MH]⁺ 259.2. Purity (LC-MS): 95.1%.

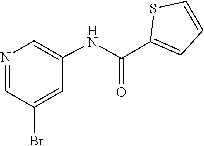 [SMILES: O=C(Nc1cncc(Br)c1)c1cccs1]

N-(5-Bromopyridin-3-yl)thiophene-2-carboxamide (39). The title product was obtained via the reaction conditions in general method A. Instead of aqueous work up, the reaction mixture was concentrated under reduced pressure, followed by purification to afford the title product as a white solid (74%). ¹H NMR (400 MHz, 2/1 CDCl₃/CD₃OD) δ 8.50 (s, 1H), 8.31 (s, 1H), 8.11 (s, 1H), 7.65 (d, J=3.2 Hz, 1H), 7.41 (d, J=5.0 Hz, 1H), 6.93 (d, J=4.0 Hz, 1H). ¹³C NMR (100 MHz, 2/1 CDCl₃/CD₃OD) δ 165.6, 148.9, 143.4, 142.5, 140.7, 135.9, 134.5, 133.4, 131.8, 124.4. [MH]⁺ 283.0 and 285.0. Purity (LC-MS): 97.2%.

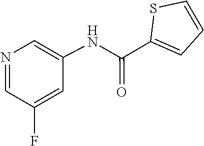 [SMILES: O=C(Nc1cncc(F)c1)c1cccs1]

N-(5-Fluoropyridin-3-yl)thiophene-2-carboxamide (40). The title product was obtained via the reaction conditions in general method A. Instead of aqueous work up, the reaction mixture was concentrated under reduced pressure, followed by purification via flash chromatography (EtOAc/hexanes 1:1) to afford the title product as a white solid (62%). ¹H NMR (400 MHz, 2/1 CDCl₃/CD₃OD) δ 165.7 (d, J=8.7 Hz), 163.4 (d, J=254.7 Hz), 142.6, 141.1 (d, J=3.8 Hz), 141.0 (d, J=3.5 Hz), 136.2, 135.9, 133.4, 131.8, 119.3 (d, J=22.9 Hz), 119.2 (d, J=22.9 Hz). ¹³C NMR (100 MHz, 2/1 CDCl₃/CD₃OD) δ 165.7 (d, J=8.7 Hz), 163.4 (d, J=254.7 Hz), 142.6, 141.1 (d, J=3.8 Hz), 141.0 (d, J=3.5 Hz), 136.2, 135.9, 133.4, 131.8, 119.3 (d, J=22.9 Hz), 119.2 (d, J=22.9 Hz). [MH]⁺ 223.1. Purity (LC-MS): 97.9%.

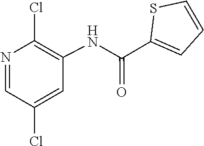 [SMILES: O=C(Nc1cc(Cl)cnc1Cl)c1cccs1]

N-(2,5-Dichloropyridin-3-yl)thiophene-2-carboxamide (41). A solution of thiophene-2-carboxylic acid (0.305 g, 2.38 mmol) in SOCl₂ (5 mL) was stirred under reflux for 3 h and then cooled to rt. After evaporation of excess SOCl₂, the residue was dissolved in CH₂Cl₂ (6 mL). To 3 mL of the above solution was added CH₂Cl₂ (7 mL), Et₃N (0.25 mL, 1.80 mmol) and 2,5-dichloropyridin-3-amine (0.165 g, 1.01 mmol). The resulting solution was stirred for 17 h at rt and was diluted with CH₂Cl₂ (30 mL), and washed with 4 N K₂CO3 solution (30 mL). The organic layer was dried over Na2SO₄, filtered and concentrated under reduced pressure. The residue was purified via flash chromatography on silica gel twice (4:1 hexane/EtOAc then pure CH₂Cl₂) to afford the title product (0.026 g, 9%) as a white solid. ¹H NMR (400 MHz, CDCl₃) δ 8.86 (d, J=2.4 Hz, 1H), 8.17 (s, 1H), 8.03 (d, J=2.4 Hz, 1H), 7.62 (dd, J=3.8, 1.1 Hz, 1H), 7.57 (dd, J=5.0, 1.1 Hz, 1H), 7.11 (dd, J=5.0, 3.8 Hz, 1H). ¹³C NMR (100 MHz, CDCl₃) δ 159.9, 142.2, 137.8, 137.4, 132.4, 132.2, 131.8, 129.4, 128.2, 128.2. [MH]⁺ 273.0 and 275.0. Purity (LC-MS): 95.4%.

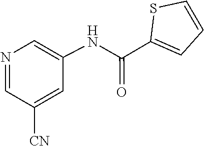 [SMILES: N#Cc1cncc(NC(=O)c2cccs2)c1]

N-(5-Cyanopyridin-3-yl)thiophene-2-carboxamide (42). The title product was obtained via the reaction conditions in general method A. Instead of aqueous work up, the reaction mixture was concentrated under reduced pressure, followed by purification via flash chromatography (EtOAc/hexanes 1:1) to afford the title product as a white solid (30%). ¹H NMR (400 MHz, 2/1 CDCl₃/CD₃OD) δ 8.82 (s, 1H), 8.44 (s, 1H), 8.35 (s, 1H), 7.71-7.64 (m, 1H), 7.48-7.41 (m, 1H), 6.98-6.90 (m, 1H). ¹³C NMR (100 MHz, 2/1 CDCl₃/CD₃OD) δ 165.7, 150.3, 148.7, 142.2, 136.2, 134.1, 134.0, 133.7, 131.9, 120.1. [MH]⁺ 230.1. Purity (LC-MS): 98.7%

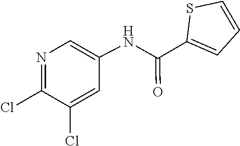 [SMILES: O=C(Nc1cnc(Cl)c(Cl)c1)c1cccs1]

N-(5,6-Dichloropyridin-3-yl)thiophene-2-carboxamide (43). The title product was obtained via the reaction conditions in general method A. Instead of aqueous work up, the reaction mixture was concentrated under reduced pressure, followed by purification via flash chromatography (EtOAc/hexanes 1:2) to afford the title product as a white solid (61%). ¹H NMR (400 MHz, 2/1 CDCl₃/CD₃OD) δ 8.33 (d, J=2.3 Hz, 1H), 8.26 (d, J=2.3 Hz, 1H), 7.64 (dd, J=3.8, 1.1 Hz, 1H), 7.41 (dd, J=5.0, 1.2 Hz, 1H), 6.92 (dd, J=5.0, 3.8 Hz, 1H). ¹³C NMR (100 MHz, 2/1 CDCl₃/CD₃OD) δ 165.5, 146.6, 142.9, 142.3, 139.6, 136.0, 134.3, 134.2, 133.5, 131.8. [MH]⁺ 273.0 and 275.0. Purity (LC-MS): 98.1%.

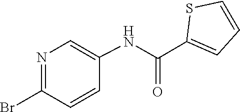 [SMILES: O=C(Nc1ccc(Br)nc1)c1cccs1]

N-(6-Bromopyridin-3-yl)thiophene-2-carboxamide (44). The title product was obtained via the reaction conditions in general method A. Instead of aqueous work up, the reaction mixture was concentrated under reduced pressure, followed by purification via flash chromatography (EtOAc/hexanes 1:1) to afford the title product as a light yellow solid (85%). 1H NMR (400 MHz, 2/1 CDCl₃/CD₃OD) δ 8.39 (d, J=2.7 Hz, 1H), 7.92 (dd, J=8.7, 2.7 Hz, 1H), 7.65 (d, J=3.9 Hz, 1H), 7.41 (d, J=4.9 Hz, 1H), 7.27 (d, J=8.6 Hz, 1H), 6.92 (t, J=4.4 Hz, 1H). ¹³C NMR (100 MHz, 2/1 CDCl₃/CD₃OD) δ 165.5, 145.8, 142.6, 139.3, 139.1, 135.7, 134.8, 133.3, 131.9, 131.8. [MH]⁺ 283.0 and 285.0. Purity (LC-MS): 98%.

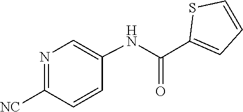 [SMILES: N#Cc1ccc(NC(=O)c2cccs2)cn1]

N-(6-Cyanopyridin-3-yl)thiophene-2-carboxamide (45). The title product was obtained via the reaction conditions in general method A. Instead of aqueous work up, the reaction mixture was concentrated under reduced pressure, followed by purification via flash chromatography (EtOAc/hexanes 1:1) to afford the title product as a white solid (85%). ¹H NMR (400 MHz, DMSO-d6) δ 10.81 (s, 1H), 9.05 (dd, J=2.5, 0.7 Hz, 1H), 8.40 (dd, J=8.6, 2.5 Hz, 1H), 8.08 (dd, J=3.8, 1.2 Hz, 1H), 8.03 (dd, J=8.6, 0.7 Hz, 1H), 7.97 (dd, J=5.0, 1.1 Hz, 1H), 7.29 (dd, J=5.0, 3.8 Hz, 1H). ¹³C NMR (100 MHz, DMSO-d6) δ 161.1, 143.2, 139.4, 139.0, 133.8, 131.0, 130.0, 128.8, 127.3, 126.8, 118.2. [MH]⁺ 230.1. Purity (LC-MS): 97.3%.

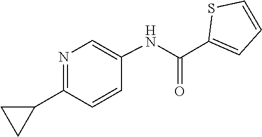 [SMILES: O=C(Nc1ccc(C2CC2)nc1)c1cccs1]

N-(6-Cyclopropylpyridin-3-yl)thiophene-2-carboxamide (46). The title product was obtained via the reaction conditions in general method A. Instead of aqueous work up, the reaction mixture was concentrated under reduced pressure, followed by purification via flash chromatography (EtOAc/hexanes 1:1) to afford the title product as a light orange solid (53%). 1H NMR (400 MHz, 2/1 CDCl₃/CD₃OD) δ 8.43-8.11 (m, 1H), 7.89-7.72 (m, 1H), 7.63-7.56 (m, 1H), 7.39-7.31 (m, 1H), 6.90-6.81 (m, 2H), 1.88-1.72 (m, 1H), 0.82-0.72 (m, 2H), 0.71-0.60 (m, 2H). ¹³C NMR (100 MHz, 2/1 CDCl₃/CD₃OD) δ 165.5, 162.4, 144.9, 142.9, 136.7, 135.3, 133.2, 133.0, 131.7, 124.6, 20.4, 13.2. [MH]⁺ 245.1. Purity (LC-MS): 98%.

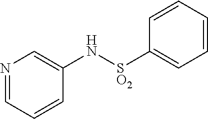 [SMILES: O=S(=O)(Nc1cccnc1)c1ccccc1]

N-(Pyridin-3-yl)benzenesulfonamide (47). A mixture of benzenesulfonyl chloride (0.415 g, 2.35 mmol), pyridine (6 mL) and pyridin-3-amine (0.193 g, 2.06 mmol) was stirred at rt. After 17 h the mixture was poured into ice-water (50 mL) to gradually form a white suspension. The suspension was filtered and the filter cake was washed with ice-cold water (200 mL). The filter cake was then dissolved in CH₂Cl₂ (200 mL) and dried over Na2SO4, filtered and concentrated under reduced pressure. The residue was purified via flash chromatography on silica gel (1:19 methanol/CH₂Cl₂) to afford the title product (0.348 g, 72%) as a white solid. 1H NMR (400 MHz, DMSO-d6) δ 10.56 (s, 1H), 8.26 (d, J=1.9 Hz, 1H), 8.23 (dd, J=4.7, 1.4 Hz, 1H), 7.80-7.70 (m, 2H), 7.65-7.58 (m, 1H), 7.59-7.49 (m, 2H), 7.48 (ddd, J=8.3, 2.7, 1.5 Hz, 1H), 7.26 (ddd, J=8.3, 4.7, 0.8 Hz, 1H). ¹³C NMR (100 MHz, DMSO-d6) δ 145.8, 142.2, 139.5, 134.8, 133.6, 129.9, 127.8, 127.1, 124.4. [MH]⁺ 235.1. Purity (LC-MS): 93.2%.

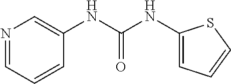 [SMILES: O=C(Nc1cccnc1)Nc1cccs1]

1-(Pyridin-3-yl)-3-(thiophen-2-yl)urea (48). A solution of 2-isocyanatothiophene (0.149 g, 1.19 mmol), Et₃N (0.21 mL, 0.151 g, 1.50 mmol) and pyridin-3-amine (0.095 g, 1.01 mmol) in CH₂Cl₂ (5 mL) was stirred at rt for 23 h. After concentration under reduced pressure, the remaining residue was dissolved in a small amount of 15% CH₃OH in CH₂Cl₂ and purified via flash chromatography on silica gel (1:19 methanol/CH₂Cl₂) to afford the title product (0.103 g, 46%) as a tan solid. ¹H NMR (400 MHz, DMSO-d6) δ 9.79 (s, 1H), 8.92 (s, 1H), 8.58 (d, J=2.6 Hz, 1H), 8.18 (dd, J=4.7, 1.4 Hz, 1H), 7.91 (d, J=8.5 Hz, 1H), 7.30 (dd, J=8.3, 4.7 Hz, 1H), 6.87 (d, J=5.5 Hz, 1H), 6.80 (dd, J=5.5, 3.7 Hz, 1H), 6.57 (dd, J=3.7, 1.4 Hz, 1H). ¹³C NMR (100 MHz, DMSO-d6) δ 152.2, 143.5, 141.1, 140.6, 136.6, 125.9, 124.7, 124.1, 116.7, 110.3. [MH]⁺ 220.1. Purity (LC-MS): 97.5%.

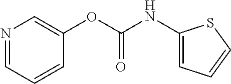 [SMILES: O=C(Nc1cccs1)Oc1cccnc1]

Pyridin-3-yl thiophen-2-ylcarbamate (49). 2-Isocyanatothiophene (0.1590 g, 1.27 mmol) was added to a stirred solution of pyridin-3-ol (0.1000 g, 1.05 mmol) in CH₂Cl₂ (5 mL) at rt. The resulting reaction mixture was stirred at rt for 17 h during which white precipitate was gradually formed. The suspension was diluted with Et₂O (15 mL) and filtered. The filter cake was washed by Et₂O and dried under high vacuum to afford the title product (0.111 g, 48%) as a white solid. ¹H NMR (400 MHz, DMSO-d6) δ 8.47 (d, J=2.7 Hz, 1H), 8.44 (dd, J=4.8, 1.4 Hz, 1H), 7.58 (d, J=7.9 Hz, 1H), 7.46 (s, 1H), 7.30 (dd, J=8.4, 4.7 Hz, 1H), 6.88 (d, J=5.5 Hz, 1H), 6.81 (dd, J=5.5, 3.7 Hz, 1H), 6.69 (s, 1H). ¹³C NMR (100 MHz, DMSO-d6) δ 151.7, 147.7, 147.2, 143.9, 140.6, 130.2, 125.3, 124.8, 117.7, 112.4. Purity (LC-MS): 79.5%.

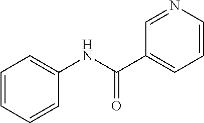 [SMILES: O=C(Nc1ccccc1)c1cccnc1]

N-Phenylnicotinamide (50). Obtained as a light brown gel according to general method A (55% yield). ¹H NMR (400 MHz, CDCl₃) δ 9.07 (d, J=2.3 Hz, 1H), 8.73 (d, J=4.8 Hz, 1H), 8.28 (s, 1H), 8.18 (d, J=8.1 Hz, 1H), 7.63 (d, J=8.0 Hz, 2H), 7.47-7.31 (m, 3H), 7.17 (t, J=7.4 Hz, 1H). ¹³C NMR (100 MHz, CDCl₃) δ 164.0, 152.4, 147.9, 137.5, 135.4, 130.8, 129.2, 125.1, 123.7, 120.5. [MH]⁺ 199.1. Purity (LC-MS): 97.1%,

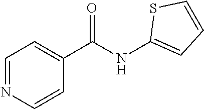 [SMILES: O=C(Nc1cccs1)c1ccncc1]

N-(Thiophen-2-yl)isonicotinamide (51). A mixture of isonicotinic acid (0.142 g, 1.15 mmol) and SOCl2 (2 mL) was stirred at reflux for 2 h. After evaporation of excess SOCl2, the isonicotinoyl chloride HCl salt was obtained and used for next step without further purification.

A mixture of tert-butyl thiophen-2-ylcarbamate (0.200 g, 1.00 mmol), CH₂Cl₂ (6 mL) and trifluoroacetic acid (TFA, 1.5 mL, 2.24 g, 19.61 mmol) was stirred at rt for 2 h. After evaporation of excess TFA and CH₂Cl₂, the thiophen-2-amine TFA salt was obtained and used as is

To a flask were added the isonicotinoyl chloride HCl salt, the thiophen-2-amine TFA salt, CH₂Cl₂ (3.5 mL), water (3.5 mL) and K2CO3 (1.2467 g, 9.03 mmol) at 0 °C. The resulting biphasic solution was warmed to rt and stirred vigorously for 21 h. The reaction mixture was diluted with brine (60 mL) and extracted with CH₂Cl₂ (2×60 mL). The combined organic layers were dried over Na₂SO₄, filtered and concentrated under reduced pressure. The residue was purified via flash chromatography on silica gel (1:19 methanol/CH₂Cl₂) to afford the title product (0.041 g, 20%) as a light yellow solid. ¹H NMR (400 MHz, CD₃OD) δ 8.77-8.67 (m, 2H), 7.94-7.82 (m, 2H), 7.04-6.98 (m, 1H), 6.97-6.94 (m, 1H), 6.94-6.86 (m, 1H). ¹³C NMR (100 MHz, CD₃OD) δ 162.4, 149.7, 141.5, 139.1, 123.8, 121.6, 118.0, 113.1. [MH]⁺ 205.1. Purity (LC-MS): 95.8%.

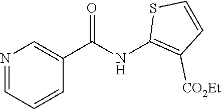 [SMILES: CCOC(=O)c1ccsc1NC(=O)c1cccnc1]

Methyl 2-(pyridine-3-amido)thiophene-3-carboxylate (52). Obtained as a yellow solid according to general method A (23% yield). ¹H NMR (400 MHz, CDCl₃) δ 12.09 (s, 1H), 9.28 (d, J=2.4 Hz, 1H), 8.82 (d, J=4.9 Hz, 1H), 8.30 (dt, J=8.0, 2.0 Hz, 1H), 7.48 (dd, J=8.1, 4.8 Hz, 1H), 7.26 (d, J=5.8 Hz, 1H), 6.83 (d, J=5.8 Hz, 1H), 3.93 (s, 3H). ¹³C NMR (100 MHz, CDCl₃) δ 166.4, 161.9, 153.3, 148.9, 148.7, 135.1, 127.9, 123.9, 123.7, 116.8, 113.5, 52.0. [MH]⁺ 263.1. Purity (LC-MS): 86.80%.

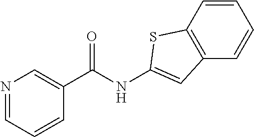 [SMILES: O=C(Nc1cc2ccccc2s1)c1cccnc1]

N-(1-Benzothiophen-2-yl)pyridine-3-carboxamide (53). Obtained as a yellow solid according to general method A (70% yield). ¹H NMR (400 MHz, DMSO-d6) δ 11.92 (s, 1H), 9.16 (d, J=2.4 Hz, 1H), 8.79 (dd, J=4.8, 1.7 Hz, 1H), 8.35 (dt, J=8.0, 2.0 Hz, 1H), 7.86 (d, J=7.9 Hz, 1H), 7.72 (d, J=7.8 Hz, 1H), 7.60 (dd, J=8.0, 4.8 Hz, 1H), 7.31 (ddd, J=8.1, 7.1, 1.2 Hz, 1H), 7.27-7.18 (m, 2H). ¹³C NMR (100 MHz, DMSO-d6) δ 163.2, 153.1, 149.3, 140.5, 137.4, 136.1, 135.2, 129.3, 124.9, 124.1, 123.4, 122.6, 122.3, 108.2. [MH]⁺ 255.1. Purity (LC-MS): 97.9%.

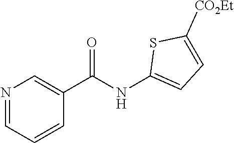 [SMILES: CCOC(=O)c1ccc(NC(=O)c2cccnc2)s1]

Methyl 5-(pyridine-3-amido)thiophene-2-carboxylate (54). Obtained as a white solid according to general method A (49% yield). ¹H NMR (400 MHz, 2:1 CD₃OD/CDCl₃) δ 9.12 (dd, J=2.3, 0.9 Hz, 1H), 8.71 (dd, J=4.9, 1.6 Hz, 1H), 8.36 (ddd, J=8.0, 2.3, 1.7 Hz, 1H), 7.64 (d, J=4.2 Hz, 1H), 7.55 (dd, J=8.0, 4.9 Hz, 1H), 6.90 (d, J=4.2 Hz, 1H), 3.85 (s, 3H). ¹³C NMR (100 MHz, 2:1 CD₃OD/CDCl₃) δ 164.0, 162.9, 152.0, 148.2, 146.3, 136.2, 131.9, 129.1, 124.0, 123.8, 113.2, 51.5. [MH]⁺ 263.1. Purity (LC-MS): 96.4%.

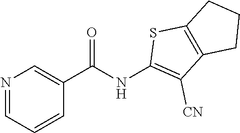 [SMILES: N#Cc1c(NC(=O)c2cccnc2)sc2c1CCC2]

N-(3-Cyano-5,6-dihydro-4H-cyclopenta[b]thiophen-2-yl)nicotinamide (55). Obtained as a light brown solid according to general method A (43% yield). ¹H NMR (400 MHz, DMSO-d6) δ 11.99 (s, 1H), 9.05 (d, J=1.5 Hz, 1H), 8.77 (dd, J=4.9, 1.6 Hz, 1H), 8.27 (dt, J=8.0, 2.0 Hz, 1H), 7.58 (dd, J=8.0, 4.8 Hz, 1H), 2.86 (dd, J=8.0, 6.6 Hz, 2H), 2.74 (t, J=7.3 Hz, 2H), 2.36 (p, J=7.2 Hz, 2H). ¹³C NMR (100 MHz, DMSO-d6) δ 164.4, 153.2, 151.2, 149.5, 142.1, 136.5, 135.6, 128.8, 124.0, 114.9, 92.4, 29.6, 28.1, 27.9. [MH]⁺ 270.1. Purity (LC-MS): 96.1%.

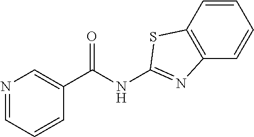 [SMILES: O=C(Nc1nc2ccccc2s1)c1cccnc1]

N-(Benzo[d]thiazol-2-yl)nicotinamide (56). Obtained as a white solid according to general method A (2% yield). ¹H NMR (400 MHz, 3:1 CDCl₃:CD₃OD) δ 9.04 (s, 1H), 8.54 (d, J=3.4 Hz, 1H), 8.26 (d, J=8.0 Hz, 1H), 7.63 (d, J=7.9 Hz, 1H), 7.53 (d, J=8.1 Hz, 1H), 7.37 (dd, J=8.0, 4.9 Hz, 1H), 7.26-7.20 (m, 1H), 7.13 (td, J=7.6, 1.2 Hz, 1H). ¹³C NMR (100 MHz, 3:1 CDCl₃:CD₃OD) δ 165.6, 152.0, 148.6, 136.6, 131.0, 129.2, 126.3, 124.0, 123.9, 121.3, 119.5, 77.2. [MH]⁺ 256.1. Purity (LC-MS): 96.2%.

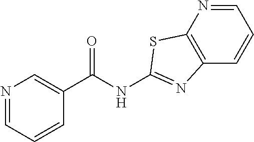 [SMILES: O=C(Nc1nc2cccnc2s1)c1cccnc1]

N-(Thiazolo[5,4-b]pyridin-2-yl)nicotinamide (57). A solution of nicotinic acid (0.133 g, 1.08 mmol), DMAP (0.030 g, 0.244 mmol), and EDC hydrochloride (0.235 g, 1.23 mmol) in DMF (0.8 mL) and CH₂Cl₂ (5 mL) was stirred at rt for 5 min followed by the addition of thiazolo[5,4-b]pyridin-2-amine (0.156 g, 1.12 mmol). The resulting solution was stirred for 44 h at rt and was filtered to remove insoluble solids. The filtrate was concentrated under reduced pressure and the residue was purified via flash chromatography on silica gel (1:9 methanol/CH₂Cl₂). The resulting compound was further purified by dissolving in 10 mL Et₂O. After sonification, and filtration, the resulting filtrate was concentrated under vacuum to afford the title product (0.063 g, 23%) as a light tan solid. ¹H NMR (400 MHz, DMSO-d6) δ 13.23 (s, 1H), 9.23 (d, J=1.5 Hz, 1H), 8.81 (dd, J=4.9, 1.6 Hz, 1H), 8.50 (dd, J=4.7, 1.5 Hz, 1H), 8.44 (dt, J=8.1, 2.0 Hz, 1H), 8.15 (d, J=8.2 Hz, 1H), 7.60 (dd, J=8.0, 4.8 Hz, 1H), 7.52 (dd, J=8.2, 4.7 Hz, 1H). ¹³C NMR (100 MHz, DMSO-d6) δ 153.8, 149.8, 146.3, 136.6, 128.3, 128.0, 124.1, 122.3. [MH]⁺ 257.1. Purity (LC-MS): 93.9%.

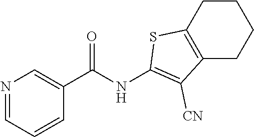 [SMILES: N#Cc1c(NC(=O)c2cccnc2)sc2c1CCCC2]

N-(3-Cyano-4,5,6,7-tetrahydrobenzo[b]thiophen-2-yl)nicotinamide (58). Obtained as a yellow solid according to general method A (48% yield). ¹H NMR (400 MHz, CDCl₃) δ 9.55 (s, 1H), 9.18 (s, 1H), 8.79 (d, J=3.4 Hz, 1H), 8.25 (dt, J=8.1, 2.0 Hz, 1H), 7.46 (dd, J=8.0, 4.8 Hz, 1H), 2.74-2.62 (m, 2H), 2.62-2.53 (m, 2H), 1.84 (d, J=5.9 Hz, 4H). ¹³C NMR (100 MHz, CDCl₃) δ 162.5, 153.3, 148.8, 146.3, 135.5, 131.4, 129.3, 128.1, 123.6, 114.5, 94.6, 24.0, 23.9, 23.0, 22.1.

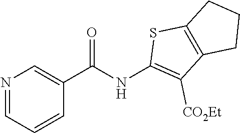 [SMILES: CCOC(=O)c1c(NC(=O)c2cccnc2)sc2c1CCC2]

Ethyl 2-(nicotinamido)-5,6-dihydro-4H-cyclopenta[b]thiophene-3-carboxylate (59). Obtained as a brown solid according to general method A (63% yield). ¹H NMR (400 MHz, CDCl₃) δ 12.04 (s, 1H), 9.19 (d, J=2.1 Hz, 1H), 8.73 (dd, J=4.8, 1.7 Hz, 1H), 8.21 (dt, J=8.0, 2.0 Hz, 1H), 7.39 (dd, J=8.0, 4.8 Hz, 1H), 4.29 (q, J=7.1 Hz, 2H), 2.95-2.75 (m, 4H), 2.39-2.19 (m, 2H), 1.32 (t, J=7.1 Hz, 3H). ¹³C NMR (100 MHz, CDCl3) δ 166.5, 161.5, 153.0, 151.1, 148.9, 141.6, 134.9, 133.1, 128.2, 123.6, 109.2, 60.7, 30.2, 28.9, 27.9, 14.3. [MH]⁺ 317.1. Purity (LC-MS): 97.7%.

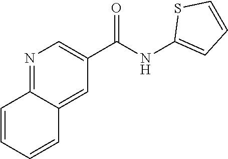 [SMILES: O=C(Nc1cccs1)c1cnc2ccccc2c1]

N-(Thiophen-2-yl)quinoline-3-carboxamide (60). A mixture of quinoline-3-carboxylic acid (0.191 g, 1.10 mmol) and SOCl₂ (2 mL) was stirred at reflux for 4 h and cooled to rt. After evaporation of excess SOCl₂, the acid chloride HCl salt was obtained and used for next step without further purification.

A mixture of tert-butyl thiophen-2-ylcarbamate (0.193 g, 0.97 mmol), CH₂Cl₂ (6 mL) and trifluoroaceetic acid (TFA, 1.5 mL, 2.24 g, 19.61 mmol) was stirred at rt for 3 h. After the evaporation of excess TFA and CH₂Cl₂, the thiophen-2-amine TFA salt was obtained.

To a flask were added the above prepared acid chloride HCl salt and the thiophen-2-amine TFA salt, CH₂Cl₂ (3.5 mL), water (3.5 mL) and K₂CO₃ (1.326 g, 9.61 mmol) at 0 °C. The resulting biphasic solution was warmed to rt and stirred vigorously for 17 h. The reaction mixture was diluted with brine (60 mL) and extracted with CH₂Cl₂ (2×60 mL). The combined organic layers were dried over Na₂SO₄, filtered and concentrated under reduced pressure. The residue was dissolved in Et₂O (20 mL) and sonicated. The precipitate was filtered and collected to afford the title product (0.045 g, 18%) as a light yellow solid. ¹H NMR (400 MHz, DMSO-d6) δ 9.44 (d, J=2.2 Hz, 1H), 8.97 (d, J=2.2 Hz, 1H), 8.14-8.04 (m, 2H), 7.84 (t, J=7.0 Hz, 1H), 7.67 (t, J=6.9 Hz, 1H), 6.94-6.84 (m, 3H). ¹³C NMR (100 MHz, DMSO-d6) δ 162.6, 150.1, 148.7, 136.0, 131.4, 129.6, 129.2, 127.7, 127.2, 124.4, 117.1, 112.5. [MH]⁺ 255.1. Purity (LC-MS): 88-9%

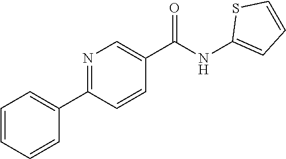 [SMILES: O=C(Nc1cccs1)c1ccc(-c2ccccc2)nc1]

6-Phenyl-N-(thiophen-2-yl)pyridine-3-carboxamide (61). A mixture of 6-phenylpyridine-3-carboxylic acid (0.225 g, 1.13 mmol) and SOCl₂ (2.5 mL) was stirred at reflux for 1 h and cooled to rt. After evaporation of excess SOCl₂, the acid chloride HCl salt was obtained and used for next step without further purification.

A mixture of tert-butyl thiophen-2-ylcarbamate (0.205 g, 1.03 mmol), CH₂Cl₂ (6 mL) and trifluoroacetic acid (TFA, 1.5 mL, 2.235 g, 19.61 mmol) was stirred at rt for 2 h. After evaporation of excess TFA and CH₂Cl₂, the thiophen-2-amine TFA salt was obtained.

To a flask were added the above prepared acid chloride HCl salt and the thiophen-2-amine TFA salt, CH₂Cl₂ (3.5 mL), water (3.5 mL) and K₂CO₃ (1.313 g, 9.51 mmol) at 0 °C. The resulting biphasic solution was warmed to rt and stirred vigorously for 19 h. The reaction mixture was diluted with brine (60 mL) and extracted with CH₂Cl₂ (2×60 mL). The combined organic layers were dried over Na₂SO₄, filtered and concentrated under reduced pressure. The residue was purified via flash chromatography on silica gel (1:19 methanol/CH₂Cl₂) followed by recrystallization (CH₂Cl₂/hexane mixture) to afford the title product (0.092 g, 32%) as a brown solid. ¹H NMR (400 MHz, CDCl₃) δ 9.46 (s, 1H), 9.17 (d, J=2.4 Hz, 1H), 8.25 (dd, J=8.3, 2.4 Hz, 1H), 8.00-7.92 (m, 2H), 7.76 (d, J=8.4 Hz, 1H), 7.53-7.39 (m, 3H), 6.93 (d, J=5.4 Hz, 1H), 6.90-6.81 (m, 2H). ¹³C NMR (100 MHz, CDCl₃) δ 160.3, 147.9, 138.8, 137.9, 136.4, 130.0, 129.0, 127.3, 127.1, 124.2, 120.4, 118.7, 113.2. [MH]⁺ 281.1. Purity (LC-MS): 91.1%.

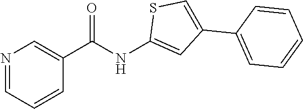 [SMILES: O=C(Nc1cc(-c2ccccc2)cs1)c1cccnc1]

N-(4-Phenylthiophen-2-yl)pyridine-3-carboxamide (62). Obtained as a yellow solid according to general method A (81% yield). ¹H NMR (400 MHz, 1:3 CD₃OD/CDCl₃) δ 8.96 (dd, J=2.3, 0.8 Hz, 1H), 8.52 (dd, J=4.9, 1.6 Hz, 1H), 8.19 (dt, J=8.0, 2.0 Hz, 1H), 7.45-7.38 (m, 2H), 7.35 (ddd, J=8.0, 4.9, 0.9 Hz, 1H), 7.21 (dd, J=8.4, 7.0 Hz, 2H), 7.14-7.05 (m, 2H), 6.92 (d, J=1.7 Hz, 1H). ¹³C NMR (100 MHz, 1:3 CD₃OD/CDCl₃) δ 162.4, 151.5, 147.9, 139.8, 138.9, 136.2, 135.7, 129.6, 128.6, 126.9, 125.9, 123.8, 113.0, 111.9. [MH]⁺ 281.1. Purity (LC-MS): 97.4%.

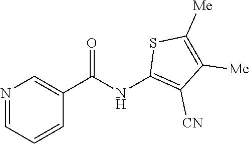 [SMILES: Cc1sc(NC(=O)c2cccnc2)c(C#N)c1C]

N-(3-Cyano-4,5-dimethylthiophen-2-yl)nicotinamide (63). A mixture of nicotinic acid (0.127 g, 1.03 mmol), DMAP (0.028 g, 0.23 mmol), EDC hydrochloride (0.22 g, 1.15 mmol) and CH₂Cl₂ (5 mL) was stirred at rt for 5 min and then 2-amino-4,5-dimethylthiophene-3-carbonitrile (0.160 g, 1.05 mmol) was added. The resulting solution was stirred for 46 h at rt and after evaporation of the solvent, the residue was dissolved in Et₂O (120 mL) and sonicated. The supernatant was removed via glass pipette and the remaining brown solid was purified via flash chromatography on silica gel (1:19 methanol/CH₂Cl₂) to afford the title product (0.092 g, 35%) as a light yellow solid. ¹H NMR (400 MHz, DMSO-d6) δ 9.04 (d, J=2.4 Hz, 1H), 8.77 (dd, J=4.8, 1.6 Hz, 1H), 8.26 (dt, J=8.0, 2.0 Hz, 1H), 7.57 (dd, J=7.9, 4.8 Hz, 1H), 2.26 (s, 3H), 2.12 (s, 3H). ¹³C NMR (100 MHz, DMSO-d6) δ 164.4, 153.2, 149.5, 145.3, 136.6, 129.7, 128.9, 126.3, 124.0, 115.1, 98.2, 12.7, 12.6. [MH]⁺ 258.1. Purity (LC-MS): 95.9%.

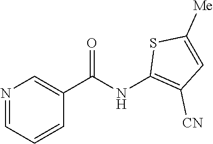 [SMILES: Cc1cc(C#N)c(NC(=O)c2cccnc2)s1]

N-(3-cyano-5-methylthiophen-2-yl)nicotinamide (64). Obtained as a yellow solid according to general method A (63% yield). ¹H NMR (400 MHz, CDCl₃:CD₃OD) δ 9.05 (d, J=2.2 Hz, 1H), 8.68 (dd, J=4.9, 1.7 Hz, 1H), 8.28 (dt, J=8.0, 1.9 Hz, 1H), 7.49 (dd, J=8.0, 4.9 Hz, 1H), 6.66 (d, J=1.3 Hz, 1H), 2.37 (d, J=1.3 Hz, 3H). ¹³C NMR (100 MHz, 2:1 CDCl₃:CD₃OD) δ 167.7, 156.2, 152.6, 150.7, 140.5, 137.4, 132.9, 127.8, 125.9, 118.5, 99.0, 18.3. [MH]⁺ 244.1. Purity (LC-MS): 95.1%.

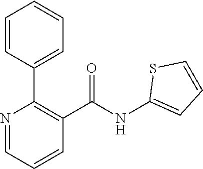 [SMILES: O=C(Nc1cccs1)c1cccnc1-c1ccccc1]

2-Phenyl-N-(thiophen-2-yl)nicotinamide (65). A mixture of 2-phenylnicotinic acid (0.100 g, 0.50 mmol) and SOCl₂ (2.5 mL) was stirred at reflux for 4 h and cooled to rt. After evaporation of excess SOCl2, the acid chloride HCl salt was obtained and used for next step without further purification.

A mixture of tert-butyl thiophen-2-ylcarbamate (0.120 g, 0.60 mmol), CH₂Cl₂ (4 mL) and trifluoroacetic acid (TFA, 1 mL, 1.490 g, 13.07 mmol) was stirred at rt for 4 h. After evaporation of excess TFA and CH₂Cl₂, the thiophen-2-amine TFA salt was obtained.

To a flask were added the above prepared acid chloride HCl salt and the thiophen-2-amine TFA salt, CH₂Cl₂ (2.5 mL), water (2.5 mL) and K₂CO₃ (0.592 g, 4.29 mmol) at 0 °C. The resulting biphasic solution was warmed to rt and stirred vigorously for 24 h. The reaction mixture was diluted with brine (60 mL) and extracted with CH₂Cl₂ (2×60 mL). The combined organic layers were dried over Na₂SO₄, filtered and concentrated under reduced pressure. The residue was purified viah flash chromatography on silica gel (1:19 methanol/DCM) to afford the title product (0.058 g, 42%) as a brown solid. ¹H NMR (400 MHz, 1:2 CDCl₃:CD₃OD) δ 8.69 (d, J=4.9 Hz, 1H), 7.98 (d, J=7.8 Hz, 1H), 7.67-7.55 (m, 2H), 7.50-7.37 (m, 4H), 6.88 (d, J=5.5 Hz, 1H), 6.79 (dd, J=5.5, 3.7 Hz, 1H), 6.56 (dd, J=3.8, 1.4 Hz, 1H). ¹³C NMR (100 MHz, 1:2 CDCl₃:CD₃OD) δ 165.6, 156.4, 150.0, 138.9, 138.4, 137.0, 131.3, 129.0, 128.4, 128.3, 123.9, 122.1, 117.8, 112.7. [MH]⁺ 281.1. Purity (LC-MS): 90.1%.

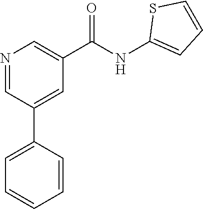 [SMILES: O=C(Nc1cccs1)c1cncc(-c2ccccc2)c1]

5-Phenyl-N-(thiophen-2-yl)nicotinamide (66). A mixture of 5-phenylnicotinic acid (0.100 g, 0.50 mmol) and SOCl₂ (2.5 mL) was stirred at reflux for 4 h and cooled to rt. After evaporation of excess SOCl₂, the acid chloride HCl salt was obtained and used for next step without further purification.

A mixture of tert-butyl thiophen-2-ylcarbamate (0.108 g, 0.54 mmol), CH₂Cl₂ (4 mL) and trifluoroacetic acid (TFA, 1 mL, 1.490 g, 13.07 mmol) was stirred at rt for 4 h. After evaporation of excess TFA and CH₂Cl₂, the thiophen-2-amine TFA salt was obtained.

To a flask were added the above prepared acid chloride HCl salt and the thiophen-2-amine TFA salt, CH₂Cl₂ (2.5 mL), water (2.5 mL) and K₂CO₃ (0.816 g, 5.91 mmol) at 0 °C. The resulting biphasic solution was warmed to rt and stirred vigorously for 18 h. The reaction mixture was diluted with brine (60 mL) and extracted with CH₂Cl₂ (2×60 mL). The combined organic layers were dried over Na₂SO₄, filtered and concentrated under reduced pressure. The residue was purified via flash chromatography on silica gel (1:9 methanol/CH₂Cl₂) to afford the title product (0.015 g, 11%) as a brown gel. ¹H NMR (400 MHz, CDCl₃) δ 9.75 (s, 1H), 9.01 (d, J=2.1 Hz, 1H), 8.84 (d, J=2.2 Hz, 1H), 8.35 (t, J=2.2 Hz, 1H), 7.49-7.42 (m, 2H), 7.42-7.28 (m, 3H), 6.86 (dd, J=4.2, 2.8 Hz, 1H), 6.80 (d, J=4.3 Hz, 2H). ¹³C NMR (100 MHz, CDCl₃) δ 162.2, 150.7, 146.2, 138.9, 137.1, 136.3, 134.1, 129.4, 129.3, 128.8, 127.2, 124.2, 118.8, 113.4. [MH]⁺281.1. Purity (LC-MS): 90.9%.

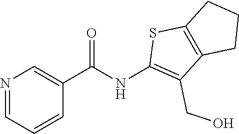 [SMILES: O=C(Nc1sc2c(c1CO)CCC2)c1cccnc1]

N-(3-(Hydroxymethyl)-5,6-dihydro-4H-cyclopenta[b]thiophen-2-yl)nicotinamide (67). To a flame-dried flask were added ethyl 2-(nicotinamido)-5,6-dihydro-4H-cyclopenta[b]thiophene-3-carboxylate (59, 0.156 g, 0.49 mmol) and anhydrous THF (6 mL) at °C, followed by addition of DIBAL-H (1 M in hexane, 1.1 mL, 1.10 mmol). The resulting reaction solution was stirred at °C for 2 h and quenched by saturated aq. NH₄Cl (30 mL). The biphasic solution was extracted with CH₂Cl₂ (3×30 mL) and the combined CH₂Cl₂ layers were dried over Na₂SO₄, filtered and concentrated under reduced pressure. The residue was purified via flash chromatography on silica gel (1:19 CH₃OH/CH₂Cl₂) to afford the title product (0.014 g, 10%) as a yellow solid. ¹H NMR (400 MHz, 3/1 CDCl₃/CD₃OD) δ 8.99-8.74 (m, 1H), 8.63-8.39 (m, 1H), 8.09 (ddt, J=10.1, 8.0, 2.0 Hz, 1H), 7.44-7.28 (m, 1H), 4.52 (d, J=10.3 Hz, 2H), 2.80-2.59 (m, 2H), 2.54-2.36 (m, 2H), 2.31-2.04 (m, 2H). ¹³C NMR (100 MHz, 3/1 CDCl₃/CD₃OD) δ 165.88, 155.66, 151.67, 145.80, 140.11, 139.72, 138.93, 133.59, 127.95, 125.96, 62.94, 33.12, 32.19, 31.52. [MH]⁺ 275.1. Purity (LC-MS): 92.6%.

TABLE 1
Preferred derivatives

| | IC₅₀ (μM) | | |
|---|---|---|---|
| | [nicotinamide] | | |
| Compound | 0 μM | 33 μM | 100 μM |
| 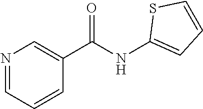 [SMILES: O=C(Nc1cccs1)c1cccnc1] | 0.006 | 1.201 | >50 |
| 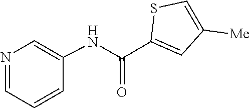 [SMILES: Cc1csc(C(=O)Nc2cccnc2)c1] | 0.302 | >50 | >50 |
| 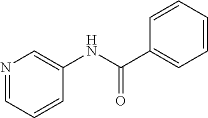 [SMILES: O=C(Nc1cccnc1)c1ccccc1] | 0.064 | 16.76 | >50 |
| 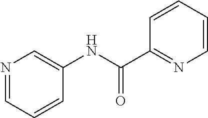 [SMILES: O=C(Nc1cccnc1)c1ccccn1] | 0.992 | >50 | >50 |
| 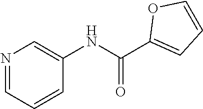 [SMILES: O=C(Nc1cccnc1)c1ccco1] | 0.022 | >50 | >50 |
| 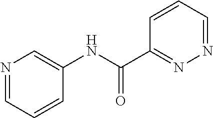 [SMILES: O=C(Nc1cccnc1)c1cccnn1] | 18.024 | >50 | >50 |
| 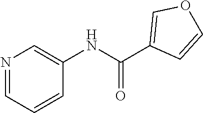 [SMILES: O=C(Nc1cccnc1)c1ccoc1] | 0.948 | >50 | >50 |
| 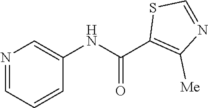 [SMILES: Cc1ncsc1C(=O)Nc1cccnc1] | >50 | >50 | >50 |
| 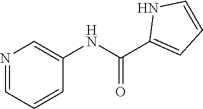 [SMILES: O=C(Nc1cccnc1)c1ccc[nH]1] | 0.080 | >50 | >50 |
| 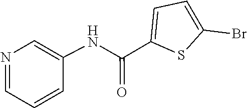 [SMILES: O=C(Nc1cccnc1)c1ccc(Br)s1] | 0.234 | 15.61 | >50 |
| 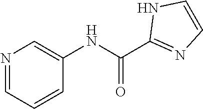 [SMILES: O=C(Nc1cccnc1)c1ncc[nH]1] | 22.376 | >50 | >50 |
| 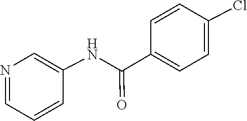 [SMILES: O=C(Nc1cccnc1)c1ccc(Cl)cc1] | 0.921 | >50 | >50 |
| 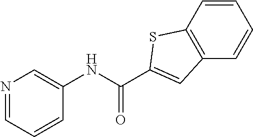 [SMILES: O=C(Nc1cccnc1)c1cc2ccccc2s1] | 0.249 | 21.390 | >50 |
| 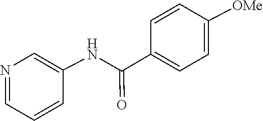 [SMILES: COc1ccc(C(=O)Nc2cccnc2)cc1] | 0.686 | >50 | >50 |
| 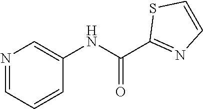 [SMILES: O=C(Nc1cccnc1)c1nccs1] | 1.875 | >50 | >50 |
| 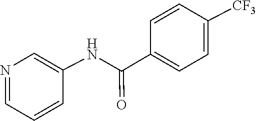 [SMILES: O=C(Nc1cccnc1)c1ccc(C(F)(F)F)cc1] | 0.771 | >50 | >50 |
| 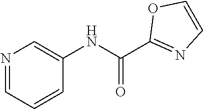 [SMILES: O=C(Nc1cccnc1)c1ncco1] | 19.451 | >50 | >50 |
| 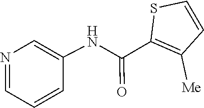 [SMILES: Cc1ccsc1C(=O)Nc1cccnc1] | 1.079 | >50 | >50 |
| 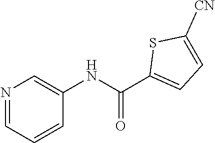 [SMILES: N#Cc1ccc(C(=O)Nc2cccnc2)s1] | 0.075 | 14.23 | >50 |
| 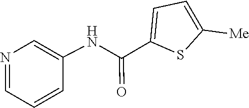 [SMILES: Cc1ccc(C(=O)Nc2cccnc2)s1] | 0.184 | >50 | >50 |
| 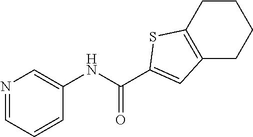 [SMILES: O=C(Nc1cccnc1)c1cc2c(s1)CCCC2] | 0.763 | >50 | >50 |
| 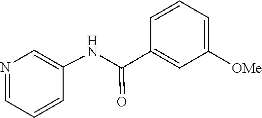 [SMILES: COc1cccc(C(=O)Nc2cccnc2)c1] | 2.356 | >50 | >50 |
| 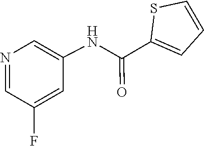 [SMILES: O=C(Nc1cncc(F)c1)c1cccs1] | >50 | >50 | >50 |
| 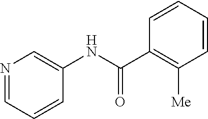 [SMILES: Cc1ccccc1C(=O)Nc1cccnc1] | 14.676 | >50 | >50 |
| 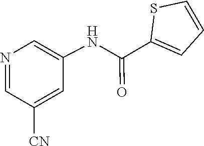 [SMILES: N#Cc1cncc(NC(=O)c2cccs2)c1] | >50 | >50 | >50 |
| 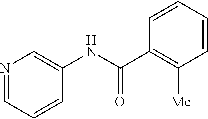 [SMILES: Cc1ccccc1C(=O)Nc1cccnc1] | >50 | >50 | >50 |
| 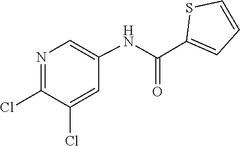 [SMILES: O=C(Nc1cnc(Cl)c(Cl)c1)c1cccs1] | 8.755 | >50 | 42.363 |
| 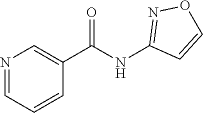 [SMILES: O=C(Nc1ccon1)c1cccnc1] | 2.733 | >50 | >50 |
| 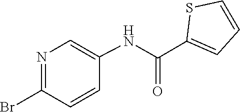 [SMILES: O=C(Nc1ccc(Br)nc1)c1cccs1] | 8.499 | >50 | >50 |
| 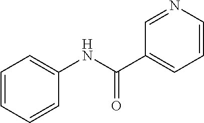 [SMILES: O=C(Nc1ccccc1)c1cccnc1] | 0.024 | 13.09 | >50 |
| 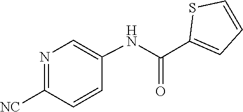 [SMILES: N#Cc1ccc(NC(=O)c2cccs2)cn1] | 2.7770 | >50 | >50 |
| 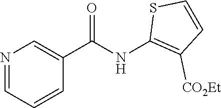 [SMILES: CCOC(=O)c1ccsc1NC(=O)c1cccnc1] | >50 | >50 | >50 |
| 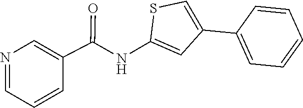 [SMILES: O=C(Nc1cc(-c2ccccc2)cs1)c1cccnc1] | 2.465 | ND | 39.632 |
| 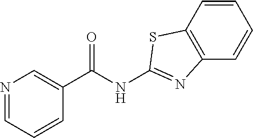 [SMILES: O=C(Nc1nc2ccccc2s1)c1cccnc1] | 0.623 | ND | >50 |
| 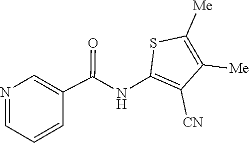 [SMILES: Cc1sc(NC(=O)c2cccnc2)c(C#N)c1C] | >50 | ND | >50 |
| 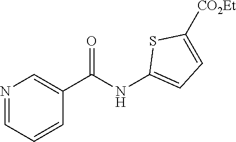 [SMILES: CCOC(=O)c1ccc(NC(=O)c2cccnc2)s1] | 0.230 | ND | >50 |
| 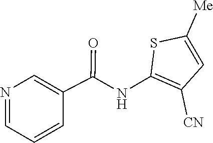 [SMILES: Cc1cc(C#N)c(NC(=O)c2cccnc2)s1] | >50 | ND | >50 |
| 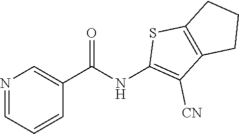 [SMILES: N#Cc1c(NC(=O)c2cccnc2)sc2c1CCC2] | 5.970 | ND | 6.098 |
| 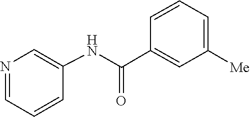 [SMILES: Cc1cccc(C(=O)Nc2cccnc2)c1] | 35.702 | >50 | >50 |
| 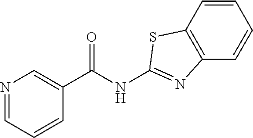 [SMILES: O=C(Nc1nc2ccccc2s1)c1cccnc1] | 36.177 | >50 | >50 |
| 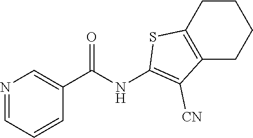 [SMILES: N#Cc1c(NC(=O)c2cccnc2)sc2c1CCCC2] | 4.744 | ND | 4.718 |
| 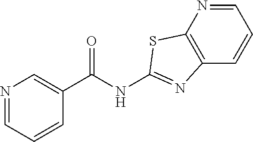 [SMILES: O=C(Nc1nc2cccnc2s1)c1cccnc1] | 23.928 | >50 | >50 |

1. A compound of formula I: 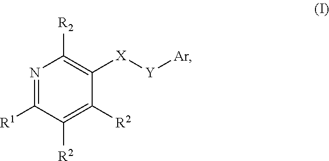
wherein:
X—Y is C(O)NH or NHC(O);
R1 is selected from H, D, F, Cl, Br, CN;
each R2 is independently selected from H or D;
Ar is selected from Ph, fused phenyl, heteroaryl, fused heteroaryl including but not limited to thiophenes, oxazoles, isoxazoles, thiazoles, isothiazoles, furans, pyrroles, pyrazoles, imidazoles, triazoles, tetrazoles, pyridines, pyrimidines, pyrazines, pyridazines, naphtyls, quinolines, isoquinolines, quinazolines, quinoxalines, cinnolines, phtalazines, naphthyridines, pyridopyrazines, pyrazolopyridines, pyridopyrimidines, pyridopyridazines, benzoxazoles, tetrahydrobenzoxazoles, benzoisoxazoles, tetrahydrobenzoisoxazoles, benzothiazoles, tetrahydrobenzothiazoles, benzoisothiazoles, tetrahydrobezoisothiazoles, indoles, tetrahydroindoles, indazoles, benzofurans, tetrahydrofurans, benzothiophenes, tetrahydrobenzothiphenes, imidazopyridines, benzodioxoles, benzoxazolones, benzoisoxazolones, benzothiazolones, benzoisothiazolones, 1,3-dihydro-2H-benzimidazoles, benzo[d]imidazol-2-ones, benzodioxolones, dioxolopyridines, oxazolopyridines, oxazolopyrimidines, oxazolopyrazines, oxazolopyridazines, isoxazolopyridines, isoxazolopyrimidines, isoxazolopyrazines, isoxazolopyridazines, thiazolopyridines, thiazolopyrimidines, thiazolopyrazines, thiazolopyridazines, imidazopyridines, thienopyridines, thienopyridazines, thienopyrimidines, thienopyrazines, furopyridines, furopyrazines, furopyrimidines, furopyridazines, pyrrolopyridines, pyrrolopyrimidines, pyrrolopirazines, pyrrolopyridazines, pyrazolopyridines, pyrazolopyrimidines, pyrazolopyrazines, pyrazolopyridazines, dioxolopyridazines, dioxolopyrimidines, dioxolopyrazines, coumarins, isocoumarins and all possible isomers thereof, each optionally substituted at any position with: D, F, Cl, Br, CN, OH, aryl, heteroaryl, R3, OR3, C(O)R3, CO2R3;
R3 is selected from C1-C3alkyl, cPr, cBu, oxetanyl, C1-C3alkynyl, C1-C3alkenyl, each optionally substituted with a substituent selected from D, F, CN, OH, OR4; and
R4 is selected from Me, CD3, CF3, CHF2, CH2F, cPr, oxetanyl;
wherein the compound and composition claims herein exclude compound 21

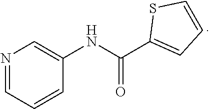 [SMILES: O=C(Nc1cccnc1)c1cccs1]

2. A compound of claim 1, wherein:
Ar is selected from Ph, thiophene, furan, pyrrole and all possible isomers thereof, each optionally substituted at any position with a substituent selected from D, F, Cl, CN, OMe, OH, OCD3, OCF3, OCHF2, OCH2F, CH2OH, CH2OMe, CH2OCD3, CH2OCF3, CH2OCHF2, CH2OCH2F.

3. A compound of claim 1, wherein:
Ar is thiophene.

4. A compound of claim 1, wherein:
R1 is H or D; and
Ar is selected from Ph, thiophene, furan, pyrrole and all possible isomers thereof, each optionally substituted at any position with a substituent selected from D, F, Cl, CN, OMe, OH, OCD3, OCF3, OCHF2, OCH2F, CH2OH, CH2OMe, CH2OCD3, CH2OCF3, CH2OCHF2, CH2OCH2F.

5. A compound of claim 1, wherein:
R1 is H or D; and
Ar is thiophene.

6. A compound of claim 1, wherein:
X—Y is C(O)NH;
R1 is H or D; and
Ar is selected from Ph, thiophene, furan, pyrrole and all possible isomers thereof, each optionally substituted at any position with a substituent selected from D, F, Cl, CN, OMe, OH, OCD3, OCF3, OCHF2, OCH2F, CH2OH, CH2OMe, CH2OCD3, CH2OCF3, CH2OCHF2, CH2OCH2F.

7. A compound of claim 1, wherein:
X—Y is C(O)NH;
R1 is H or D; and
Ar is thiophene.

8. A compound of claim 1, wherein:
X—Y is C(O)NH;
R1 is H or D; and
Ar is thiophen-2-yl.

9. A compound of claim 1, selected from Table 1.

10. A compound of claim 1 selected from:

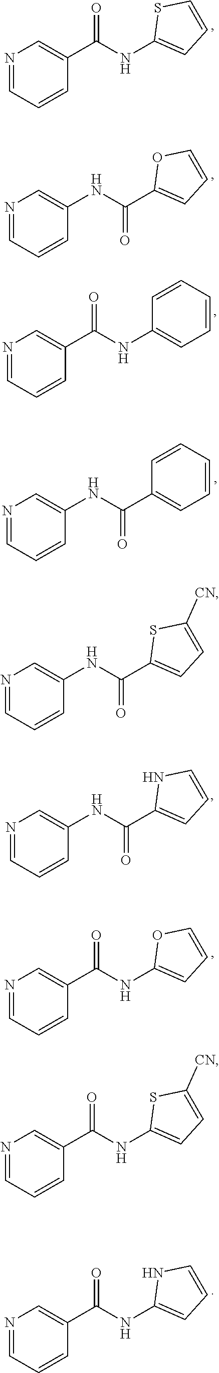

11. A compound of claim 1 of formula:

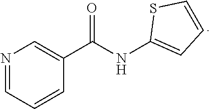 [SMILES: O=C(Nc1cccs1)c1cccnc1]

12. A pharmaceutical composition comprising a compound of claim 1, or a pharmaceutically acceptable salt, a hydrate or a stereoisomer thereof, and a pharmaceutically acceptable carrier or excipient, in a pharmaceutically acceptable unit dosage.

13. A method to treat or inhibit cancer, particularly a neuronal cancer, including peripheral and central nervous system cancers, such as glioblastoma and malignant peripheral nerve sheath tumors (MPNST), comprising administering to a person in need thereof a compound of claim 1.

14. A method of claim 13, further comprising the antecedent step of detecting or diagnosing a disease or condition indicating the need thereof.

15. A method of claim 13, further comprising the antecedent step of detecting or diagnosing a disease or condition indicating the need thereof, and the subsequent step of detecting a resultant improvement or delay of progression of the disease or condition.
